(19) United States
(12) Patent Application Publication
(10) Pub. No.: US 2011/0236397 A1
(43) Pub. Date: Sep. 29, 2011

(54) LIMITED PROTEOLYSIS OF CD2AP AND PROGRESSION OF RENAL DISEASE

(75) Inventors: Jochen Reiser, Miami, FL (US); Brian Adair, Malden, MA (US)

(73) Assignees: University of Miami, Miami, FL (US); Massachusetts General Hospital, Boston, MA (US)

(21) Appl. No.: 13/127,839

(22) PCT Filed: Nov. 6, 2009
(86) PCT No.: PCT/US09/63511

§ 371 Date: May 18, 2011

Related U.S. Application Data
(60) Provisional application No. 61/111,869, filed on Nov. 6, 2008.

(51) Int. Cl. A61K 39/395; A61K 38/48; A61K 31/7088; A61K 38/02; A61K 38/43; C12Q 1/37; C12Q 1/02; C07K 14/47; C12N 15/63; C07K 16/18; C12N 9/00; C12N 9/64; C12N 9/14; A61P 13/12; A61P 3/10; A61P 7/00; A61P 35/00; A61P 31/00

(52) U.S. Cl. 424/158.1; 424/94.65; 514/44 R; 514/20.2; 514/44 A; 514/1.1; 424/94.1; 435/23; 435/29; 530/350; 435/320.1; 530/389.8; 530/387.9; 435/183; 435/226; 435/195

(57) ABSTRACT

Compositions which specifically block cathepsin L function in podocytes, compositions which protect cytoskeletal adaptor protein (CD2AP) for degradation, compositions which modulate expression or function of cytoskeletal adaptor protein (CD2AP), protect against renal diseases or disorders. Methods of treatment in vivo involve use of one or more compositions.

### CROSS-REFERENCE TO RELATED APPLICATIONS

The present application claims the priority of U.S. provisional patent application No. 61/111,869 filed Nov. 6, 2008 which is incorporated herein by reference in its entirety.

### FIELD OF THE INVENTION

Embodiments of the invention comprise compositions which modulate expression, function, activity of cathepsin L in podocytes. Compositions which inhibit degradation and/or increase expression or activity of cytoskeletal adaptor protein (CD2AP) are also provided.

### BACKGROUND

Cathepsins are a family of enzymes that are part of the papain superfamily of cysteine proteases and include Cathepsins B, H, L, N and S. Cathepsins function in the normal physiological process of protein degradation in animals, including humans, e.g., in the degradation of connective tissue. However, elevated levels of these enzymes in the body can result in pathological conditions leading to disease. Thus, cathepsins have been implicated as causative agents in various disease states, including but not limited to, infections by Pneumocystis carinii, Trypsanoma cruzi, Trypsanoma brucei brucei, and Crithidia fusiculata; as well as in schistosomiasis, malaria, tumor metastasis, metachromatic leukodystrophy, muscular dystrophy, amytrophy, and the like.

### SUMMARY

This Summary is provided to present a summary of the invention to briefly indicate the nature and substance of the invention. It is submitted with the understanding that it will not be used to interpret or limit the scope or meaning of the claims.

Embodiments of the invention are directed to compositions for the treatment renal diseases or disorders, such as for example, proteinuria.

Other aspects are described infra.

### BRIEF DESCRIPTION OF THE DRAWINGS

FIG. 1A is a graph showing cathepsin L (CatL) activity in soluble fractions from isolated glomeruli of normal and lipopolysaccharide (LPS) treated mice. FIG. 1B is an immunoblot of soluble and pelleted fractions of the glomeruli from wild type (WT) mice. FIG. 1C is an immunoblot for anti-N-CD2AP with glomeruli from LPS-treated WT and CatL KO mice. Blots were processed with Image J software (rsb.info.nih.gov/ij) to quantify the intensities of the bands (Ratio: CD2AP/GAPDH; *P<0.0001, t test, n=5). FIG. 1D is a photograph showing the immunofluorescent labeling of WT and CatL KO mouse glomeruli against anti-N-CD2AP before and after LPS.

FIG. 2A is a scan of a photograph showing a silver stained gel of FLAG-CD2AP after cleavage with CatL at various pH. FIG. 2B is a scan of a photograph showing an immunoblot of cleaved CD2AP fragments that are tagged with a N-terminal GFP. GFP-CD2AP is stable in the absence of CatL enzyme. Cleavage of CD2AP at pH 4.5 and 5.5 leads to complete digestion of the protein. At pH 7.0, CD2AP is cleaved into a stable 55 kD fragment (▪) as detected with an anti-GFP antibody. The same fragment is detected with the CD2AP antiserum which is raised against the SH3 domains of CD2AP (anti-N-CD2AP). The CD2AP antiserum also detects a weak band corresponding to a 44 kD fragment (▴, detected by the affinity of the antibody to the C-terminal SH3, anti-C-CD2AP). FIG. 2C is a schematic representation showing the match of cleavage fragments with predicted CatL cleavage site QPLGS. FIG. 2D is a blot showing that CatL cleaves CD2AP-FLAG yielding a 44 kD (▴) and a 32 kD () fragment. FIG. 2E is a schematic representation showing that both fragments have corresponding predictions in the amino acid sequence of CD2AP (QPLGS and LSAAE). FIG. 2F is a blot showing results from a cellular CatL cleavage assay: Wild type (WT) CatL (pre-pro CatL and short form) cleaves CD2AP in HEK293 cells. This cleavage can be prevented by the incubation of the cells with a specific CatL inhibitor. Short CatL (CatL M1) is sufficient to cleave CD2AP yielding a 32 kD fragment (). This cleavage is abrogated by a specific CatL inhibitor. FIG. 2G is a blot showing that deletion of the CatL cleavage site LSAAE protects CD2AP from limited proteolysis into the 32 kD fragment ().

FIG. 3A is a photograph of a native gel (left) and CD2AP multimerization after chemical crosslinking (right). FIG. 3B is a projection histogram displaying the number of particles at particular ‘Φ’, ‘Θ’ Euler angles for the final round (top, left). Each circle represents a specific projection, and the grayscale is proportional to the number of particles belonging to that class. The scale ranges between 0 and 100 and has been truncated at the latter range. Although there are particles assigned to each class, there is a clear preferential orientation where the molecule favors placing the face with the three SH3 domains in proximity to the carbon substrate. Examples of particles from the final round of refinement (top, right). Raw particle images are displayed on the left, class averages in the middle, and back projections form the final 3D map on the right. The angular rotation of the map projection, in degrees, is indicated to the left of the particles. Fourier shell correlation to determine resolution of the final map (bottom). The entire data set was divided randomly into two equal groups. The raw particles in each class were independently aligned with one another to generate new class averages, from which two new maps were generated. The figure plots the correlation between the two maps as resolution shells in Fourier space. A correlation coefficient of 0.5 has been used to establish the resolution of the refinement (dotted lines). FIGS. 3C-3E show surface-shaded, three-dimensional density map of recombinant CD2AP at ˜21 Å resolution oriented in various directions. FIG. 3C shows a view of the map with the four-fold axis oriented in the y direction. The approximate locations of the CatL cleavage sites at positions 247 (between the second and third SH3 domains) and 352 (following the third SH3 domain) are indicated with asterisks. FIG. 3D shows the same view as in FIG. 3E but rotated about the four-fold axis. FIG. 3E shows a view of the map with upper portion of the molecule rotated toward the viewer. Assignment of the coiled-coil domain is indicated with the legend C-C. FIGS. 3F-3H show segmentation and domain assignments in the CD2AP map. FIG. 3F shows the structures of CD2AP and homologous domains positioned within the CD2AP map. The protein structures, represented with ribbon diagrams, are 1) the N-terminal SH3 domain from CD2AP in yellow, 2) the second SH3 domain from CIN85 in blue, 3) the third SH3 domain from CD2AP in green, and 4) a tetrameric GCN4 mutant coiled-coil domain in red. FIG. 3H shows the same fit as in FIG. 3E in but with the upper portion of the molecule rotated toward the viewer. FIG. 3F shows the segmentation of an individual subunit within the CD2AP map. The central core domains, indicated with red (coiled-coil domain) and violet (proline-rich and nephrin binding domains), possess extensive contacts with their symmetry-related counterparts. FIGS. 3I, 3J show the cathepsin L access to cleavage sites. The structure of human CatL has been modeled with its active site accessing the identified cleavage sites on CD2AP. Two molecules of CatL are shown, one at each site. FIGS. 3I and 3J show ribbon and surface representations of CatL, respectively. CatL has unimpeded access to each of the sites (cathepsin L at positions 247 and 352 are depicted with green and cyan colors, respectively). FIG. 3K is a representation of the C-terminal CD2AP core after CatL limited proteolysis (colored piece shows the CD2AP monomer after proteolysis). Segments corresponding to the arm domains have been computationally subtracted from the map.

FIG. 4A shows the co-immunoprecipitation of the slit diaphragm protein nephrin, synaptopodin as well as dendrin from HEK 293 cells that were transfected with N-terminal, C-terminal and full length CD2AP. FIG. 4B shows the results from the double immunofluorescent labeling for dendrin and podocyte cell marker WT-1 in wild type (WT) and CD2AP KO (5 weeks) mice. FIG. 4C shows the dendrin staining in WT, CD2AP KO and CatL knockdown podocytes. FIG. 4D shows the immunofluorescent staining of WT and CatL KO mouse glomeruli with dendrin (green) and 4′,6-diamidino-2-phenylindole (DAPI, blue) before and 14 days after serum nephritis (SN) injection. Arrows indicate the podocytes with nuclear dendrin. The quantitative analysis (right) showed that 35.0±8.6% WT, SN cells displayed nuclear dendrin versus 12.3±3.8% WT, CON cells (*P=0.0004, t test, n=10) and 16.0±2.9% CatL KO, SN cells (**P=0.0008, t test, n=10). FIG. 4E shows the immunofluorescent staining of WT mouse glomeruli with CatL and synaptopodin before and 14 days after serum nephritis (SN) injection. FIG. 4F: specificities of N- and C-terminal CD2AP antibodies detected by the immunoblots of HEK293 cells which were transfected with N-terminal, C-terminal and full length CD2AP(CON: untransfected) (top panel). Immunofluorescent staining of WT and CatL KO mouse glomeruli with N-terminal (bottom, left panel) and C-terminal CD2AP (bottom, right panel) before and 14 days after SN injection. CD2AP KO mouse glomeruli were stained as control. Staining intensities were quantified for each glomerulus using Image J software (*P<0.0001, t test, n=10).

FIG. 5A shows the histology of glomeruli in WT and CatL KO mice 14 days after SN injection. Hematoxylin and Eosin (H&E) stainings (original magnification×400) demonstrate loss of podocytes (arrows) within WT mouse glomeruli 14 days after inducing SN compared with the control whereas glomeruli from CatL KO mice do not show significant differences. FIG. 5B: The methenamine silver stain (original magnification×400) shows the loss of capillary structure, crescent formation and matrix accumulation in WT glomerulus. The podocytes that cover these segments present hypertrophy and hyperplasia. FIG. 5C: Histopathologic injury scores for kidneys from WT and CatL KO mice injected with SN. Abnormal glomerular architecture was commonly observed in WT mice characterized by hypercellularity (HC), focal segmental glomerulosclerosis (FSGS), crescent cell formation (CRES), and podocyte apoptosis (PodAP) (all *P, **P, ***P<0.0001, t test, n=30).

FIG. 6A shows immunofluorescent staining using N- and C-terminal CD2AP antibodies in kidney biopsies from patients with Minimal Change Disease and Focal Segmental Glomerulosclerosis. N-terminal CD2AP is reduced only in progressive disease (FSGS). FIG. 6B shows the expression of FLAG-CD2AP and FLAG-CD2AP with mutated cathepsin L cleavage site (FLAG-CD2AP-CatMut) in kidney of serum nephritis wild type mice. Anti-FLAG immunoprecipitation showed a prominent band at 160 kD consistent with a CD2AP dimer. Incubation with N-terminal CD2AP antisera also showed monomeric CD2AP. FIG. 6C shows decreased expression of wild type CD2AP but not of cathepsin L cleavage resistant CD2AP (CD2AP-CatMut) during serum nephritis in podocytes using double immunofluorescent labeling during serum nephritis in podocytes using double immunofluorescent labeling with synaptopodin. N-CD2AP staining intensities were quantified for both glomerulus using Image J software (*P<0.0001, t test, n=10). FIG. 6D: Phenotypic analysis of wild type mice during serum nephritis that express full wild type CD2AP or the cathepsin L resistant form, CD2AP-CatMut. H&E staining shows less glomerular damage in mice expressing the cathepsin L cleavage mutant of CD2AP. Silver stain shows prominent crescents in glomeruli where CD2AP is degraded but not in glomeruli that express CD2AP-CatMut. FIG. 6E: Semi-quantitative scores of serum nephritis wild type mice that have received wild type CD2AP or cleavage resistant CD2AP (refer to FIG. 5C for the histological lesions located along the x-axis; all *P, **P, ***P, ****P<0.0001, t test, n=30).

FIG. 7A: shows immunofluorescent staining for CatL and CD2AP in glomeruli of puromycin (PAN) treated rats (CON: untreated; d: day). FIG. 7B: immunofluorescent labeling of mouse glomeruli after gene delivery of HA-tagged cathepsin L that encodes for pre-pro cathepsin L (CatL M55-110, long) or cytosolic cathepsin L (CatL M1, short). Gene delivery of cytosolic and lysosomal forms of cathepsin L were performed into wild type mice. Reduction of CD2AP staining was found in glomeruli of animals expressing cytosolic cathepsin L but not in podocytes overexpressing lysosomal cathepsin L (arrows; CON: untransfected). FIG. 7C: shows an immunoblot for CD2AP in cultured podocytes that were exposed to LPS or PAN.

FIG. 8 shows the phosphorus NMR spectra for untreated and LPS-treated wild type (WT) podocytes. Podocytes were cultured and treated with LPS. Eighty to hundred million cells were harvested and resuspended in 2-2.5 mL of phosphate-free RPMI medium (MP Biomedicals) with glutamine (Gibco) prior to assay. Phosphorous NMR spectra were acquired on a 14 Tesla Bruker Avance NMR spectrometer (Bruker Biospin) with a 10 mm broadband observe (BBO) NMR probe. Cell suspensions were placed in 10 mm (od) glass NMR tubes (Wilmad). Samples were maintained at a temperature of 37° C. Spectra were acquired with a recycle delay time of 2 sec and consisted of 1024 averages. Spectra were analyzed using the iNMR software package (Mestrelab Research). Intracellular pH (pHi) was calculated from the chemical shift difference (d) between the intracellular inorganic phosphate peak (Pi) and the primary phosphate of nucleoside phosphates (Pa) using equation 1.

$${pH}_{i} = 6.82 + log ( \frac{d - 11.58}{13.51 - d} ) \qquad Eq . 1$$

A reference sample containing 2.2 mM disodium phosphate (RPMI-1640 medium, Gibco) and 10 mM ATP (Sigma) was used to calibrate the pHi equation. The pH was varied from 6-8 and the dependence of the chemical shift difference (d) between the inorganic phosphate peak and the alpha-phosphate peak of ATP (Pa) were fit to obtain the constants of equation 1.

FIG. 9 shows the assessment of clathrin-mediated endocytosis in HeLa cells after expression of N-terminal, C-terminal and full length CD2AP.

FIG. 10A shows the urinary albumin:creatinine ratio (ALB/CREA) in wild type (WT) and CatL KO mice 7 and 14 days after serum nephritis (SN) injection. Significant increases in ALB/CREA ratios was observed in both WT and CatL KO mice 14 days after SN injection when compared to the controls (both *P, **P<0.0001, t test, n=5). FIG. 10B shows the expression levels of synaptopodin and dynamin in WT mice after serum nephritis (SN) injection.

FIG. 11 shows trichrome stain showing crescent formation (asterisk) in a WT glomerulus after serum nephritis (original magnification×400). Occasional podocyte bridging was observed in CatL KO mice with serum nephritis, 14 days (arrow).

FIG. 12 is a schematic representation of CatL mRNA containing several AUG codons and resulting proteins. After translation from the first AUG, CatL is processed to yield a 30-kDa lysosomal form, called single-chain CatL (black arrows). However, alternative translation initiation from a downstream AUG produces a CatL isoform devoid of the lysosomal targeting sequence (short CatL), which localizes to the cytoplasm (red arrow).

FIGS. 13A-13B: CatL is important for the development of proteinuria in the LPS model. In WT mice, intraperitoneal injection of LPS leads to a T- and B-cell independent transient form of proteinuria through the activation of podocyte TLR-4 and induction of B7-1. FIG. 13A: Immunocytochemistry of mouse glomeruli using monoclonal anti-CatL antibody. WT mice receiving LPS (WT LPS) upregulate the expression of cytosolic CatL as compared to control mice receiving PBS (WT CON). LPS was also injected into CatL^{-/-} mice (CatL^{-/-} LPS). Original magnification, ×400. FIG. 13B: Electron micrographs of Fps showing effacement in LPS treated WT but not in CatL^{-/-} mice.

FIGS. 14A-14C: CatL is mRNA and protein expression are elevated in human proteinuric kidney diseases. FIG. 14A: Quantitative rt-PCR of microdissected glomeruli from human biopsies of patients with acquired proteinuric diseases: minimal change disease (MCD; n=7), membranous glomerulonephritis (MGN; n=9), focal segmental glomerulosclerosis (FSGS; n=7), and diabetic nephropathy (DN; n=10). **P<0.01 for comparison with healthy controls (CON; n=8). FIG. 14B: CatL labeling of normal human kidney. FIG. 14C: CatL labeling of a kidney biopsy from a patient with diabetic nephropathy, mildly reduced renal function, and nephrotic range proteinuria.

FIG. 15A-15D: CatL is induced in podocytes during FP effacement. FIG. 15A: In control mice, CatL expression is located mainly in lysosomes of primary podocyte processes (dashed arrow). Only few gold labeling is found in FP (solid arrows). FIG. 15B: LPS treatment induced FP effacement and induction of CatL in lysosomes of primary processes (dashed arrow) and in effaced podocyte FP (solid arrows); P: podocyte; GBM: glomerular basement membrane; END: endothelial cells; ERY: erythrocyte. FIGS. 15C, 15D: Schematic illustration of FP effacement and proteinuria as a podocyte enzymatic disease. Under normal conditions, synaptopodin and dynamin are involved in regulating podocyte F-actin. A small portion of CatL is in the cytosol and participates in a physiological turnover of synaptopodin and dynamin. The induction of cytosolic CatL causes proteolysis of synaptopodin and dynamin, thereby disrupting actin organization, causing podocyte FP effacement and proteinuria.

### DETAILED DESCRIPTION

Embodiments of the present invention relates to discoveries involving agents which modulate and/or inhibit the enzymatic activity of cathepsin L. Other agents include those which inhibit the degradation of CD2AP, and/or inhibit the rate of degradation of CD2AP. Embodiments include compositions which regulate the pH of podocytes, regulate cathepsin L activity, methods of use thereof and methods of delivery thereof. Embodiments further relate to the regulation of pathways by cathepsin L, by modulation of molecules on which cathepsin L interacts with directly or indirectly, e.g. CD2AP. Accordingly, the methods of the present invention can be used to treat disorders characterized by proteinuria.

Several aspects of the invention are described below with reference to example applications for illustration. It should be understood that numerous specific details, relationships, and methods are set forth to provide a full understanding of the invention. One having ordinary skill in the relevant art, however, will readily recognize that the invention can be practiced without one or more of the specific details or with other methods. The present invention is not limited by the illustrated ordering of acts or events, as some acts may occur in different orders and/or concurrently with other acts or events. Furthermore, not all illustrated acts or events are required to implement a methodology in accordance with the present invention.

All genes, gene names, and gene products disclosed herein are intended to correspond to homologs from any species for which the compositions and methods disclosed herein are applicable. Thus, the terms include, but are not limited to genes and gene products from humans and mice. It is understood that when a gene or gene product from a particular species is disclosed, this disclosure is intended to be exemplary only, and is not to be interpreted as a limitation unless the context in which it appears clearly indicates. Thus, for example, for the genes disclosed herein, which in some embodiments relate to mammalian nucleic acid and amino acid sequences are intended to encompass homologous and/or orthologous genes and gene products from other animals including, but not limited to other mammals, fish, amphibians, reptiles, and birds. In preferred embodiments, the genes or nucleic acid sequences are human.

### DEFINITIONS

The terminology used herein is for the purpose of describing particular embodiments only and is not intended to be limiting of the invention. As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising.”

The term “about” or “approximately” means within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value. Where particular values are described in the application and claims, unless otherwise stated the term “about” meaning within an acceptable error range for the particular value should be assumed.

As used herein, the term “safe and effective amount” or “therapeutic amount” refers to the quantity of a component which is sufficient to yield a desired therapeutic response without undue adverse side effects (such as toxicity, irritation, or allergic response) commensurate with a reasonable benefit/risk ratio when used in the manner of this invention. By “therapeutically effective amount” is meant an amount of a compound of the present invention effective to yield the desired therapeutic response. The specific safe and effective amount or therapeutically effective amount will vary with such factors as the particular condition being treated, the physical condition of the patient, the type of mammal or animal being treated, the duration of the treatment, the nature of concurrent therapy (if any), and the specific formulations employed and the structure of the compounds or its derivatives.

As used herein “proteinuria” refers to any amount of protein passing through a podocyte that has suffered podocyte damage or through a podocyte mediated barrier that normally would not allow for any protein passage. In an in vivo system the term “proteinuria” refers to the presence of excessive amounts of serum protein in the urine. Proteinuria is a characteristic symptom of either renal (kidney), urinary, pancreatic distress, nephrotic syndromes (i.e., proteinuria larger than 3.5 grams per day), eclampsia, toxic lesions of kidneys, and it is frequently a symptom of diabetes mellitus. With severe proteinuria general hypoproteinemia can develop and it results in diminished oncotic pressure (ascites, edema, hydrothorax).

The phrase “specifically binds to”, “is specific for” or “specifically immunoreactive with”, when referring to an antibody refers to a binding reaction which is determinative of the presence of the protein in the presence of a heterogeneous population of proteins and other biologics. Thus, under designated immunoassay conditions, the specified antibodies bind to a particular protein and do not bind in a significant amount to other proteins present in the sample. Specific binding to a protein under such conditions may require an antibody that is selected for its specificity for a particular protein.

As used herein, the term “aptamer” or “selected nucleic acid binding species” shall include non-modified or chemically modified RNA or DNA. The method of selection may be by, but is not limited to, affinity chromatography and the method of amplification by reverse transcription (RT) or polymerase chain reaction (PCR).

As used herein, “modulation” means either an increase (stimulation) or a decrease (inhibition) in the expression, in vivo amounts of a gene. This includes any amounts in vivo, functions and the like as compared to normal controls. The term includes, for example, increased, enhanced, increased, agonized, promoted, decreased, reduced, suppressed blocked, or antagonized. Modulation can increase activity or amounts more than 1-fold, 2-fold, 3-fold, 5-fold, 10-fold, 100-fold, etc., over baseline values. Modulation can also decrease its activity or amounts below baseline values.

The term “variant,” when used in the context of a polynucleotide sequence, may encompass a polynucleotide sequence related to a wild type gene. This definition may also include, for example, “allelic,” “splice,” “species,” or “polymorphic” variants. A splice variant may have significant identity to a reference molecule, but will generally have a greater or lesser number of polynucleotides due to alternate splicing of exons during mRNA processing. The corresponding polypeptide may possess additional functional domains or an absence of domains. Species variants are polynucleotide sequences that vary from one species to another. Of particular utility in the invention are variants of wild type gene products. Variants may result from at least one mutation in the nucleic acid sequence and may result in altered mRNAs or in polypeptides whose structure or function may or may not be altered. Any given natural or recombinant gene may have none, one, or many allelic forms. Common mutational changes that give rise to variants are generally ascribed to natural deletions, additions, or substitutions of nucleotides. Each of these types of changes may occur alone, or in combination with the others, one or more times in a given sequence.

The resulting polypeptides generally will have significant amino acid identity relative to each other. A polymorphic variant is a variation in the polynucleotide sequence of a particular gene between individuals of a given species. Polymorphic variants also may encompass “single nucleotide polymorphisms” (SNPs) or single base mutations in which the polynucleotide sequence varies by one base. The presence of SNPs may be indicative of, for example, a certain population with a propensity for a disease state, that is susceptibility versus resistance.

Derivative polynucleotides include nucleic acids subjected to chemical modification, for example, replacement of hydrogen by an alkyl, acyl, or amino group. Derivatives, e.g., derivative oligonucleotides, may comprise non-naturally-occurring portions, such as altered sugar moieties or inter-sugar linkages. Exemplary among these are phosphorothioate and other sulfur containing species which are known in the art. Derivative nucleic acids may also contain labels, including radionucleotides, enzymes, fluorescent agents, chemiluminescent agents, chromogenic agents, substrates, cofactors, inhibitors, magnetic particles, and the like.

A “derivative” polypeptide or peptide is one that is modified, for example, by glycosylation, pegylation, phosphorylation, sulfation, reduction/alkylation, acylation, chemical coupling, or mild formalin treatment. A derivative may also be modified to contain a detectable label, either directly or indirectly, including, but not limited to, a radioisotope, fluorescent, and enzyme label.

As used herein, the term “fragment or segment”, as applied to a nucleic acid sequence, gene or polypeptide, will ordinarily be at least about 5 contiguous nucleic acid bases (for nucleic acid sequence or gene) or amino acids (for polypeptides), typically at least about 10 contiguous nucleic acid bases or amino acids, more typically at least about 20 contiguous nucleic acid bases or amino acids, usually at least about 30 contiguous nucleic acid bases or amino acids, preferably at least about 40 contiguous nucleic acid bases or amino acids, more preferably at least about 50 contiguous nucleic acid bases or amino acids, and even more preferably at least about 60 to 80 or more contiguous nucleic acid bases or amino acids in length. “Overlapping fragments” as used herein, refer to contiguous nucleic acid or peptide fragments which begin at the amino terminal end of a nucleic acid or protein and end at the carboxy terminal end of the nucleic acid or protein. Each nucleic acid or peptide fragment has at least about one contiguous nucleic acid or amino acid position in common with the next nucleic acid or peptide fragment, more preferably at least about three contiguous nucleic acid bases or amino acid positions in common, most preferably at least about ten contiguous nucleic acid bases amino acid positions in common.

The terms “biomolecule” or “markers” are used interchangeably herein and refer to DNA, RNA (including mRNA, rRNA, tRNA and tmRNA), nucleotides, nucleosides, analogs, polynucleotides, peptides and any combinations thereof.

“Expression/amount” of a gene, biomolecule, or biomarker in a first sample is at a level “greater than” the level in a second sample if the expression level/amount of the gene or biomarker in the first sample is at least about 1 time, 1.2 times, 1.5 times, 1.75 times, 2 times, 3 times , 4 times, 5 times, 6 times, 7 times, 8 times, 9 times, 10 times, 20 times, 30 times, the expression level/amount of the gene or biomarker in the second sample or a normal sample. Expression levels/amounts can be determined based on any suitable criterion known in the art, including but not limited to mRNA, cDNA, proteins, protein fragments and/or gene copy. Expression levels/amounts can be determined qualitatively and/or quantitatively.

The terms “detecting”, “detect”, “identifying”, “quantifying” includes assaying, quantitating, imaging or otherwise establishing the presence or absence of the transcriptomic biomarker, or combinations of biomolecules comprising the biomarker, and the like, or assaying for, imaging, ascertaining, establishing, or otherwise determining the prognosis and/or diagnosis of renal diseases, disorders or conditions.

“Patient” or “subject” refers to mammals and includes human and veterinary subjects.

As used herein “a patient in need thereof” refers to any patient that is affected with a disorder characterized by proteinuria. In one aspect of the invention “a patient in need thereof refers to any patient that may have, or is at risk of having a disorder characterized by proteinuria.

As used herein, the term “test substance” or “candidate therapeutic agent” or “agent” are used interchangeably herein, and the terms are meant to encompass any molecule, chemical entity, composition, drug, therapeutic agent, chemotherapeutic agent, or biological agent capable of preventing, ameliorating, or treating a disease or other medical condition. The term includes small molecule compounds, antisense reagents, siRNA reagents, antibodies, enzymes, peptides organic or inorganic molecules, natural or synthetic compounds and the like. A test substance or agent can be assayed in accordance with the methods of the invention at any stage during clinical trials, during pre-trial testing, or following FDA-approval.

As used herein the phrase “diagnostic” means identifying the presence or nature of a pathologic condition. Diagnostic methods differ in their sensitivity and specificity. The “sensitivity” of a diagnostic assay is the percentage of diseased individuals who test positive (percent of “true positives”). Diseased individuals not detected by the assay are “false negatives.” Subjects who are not diseased and who test negative in the assay are termed “true negatives.” The “specificity” of a diagnostic assay is 1 minus the false positive rate, where the “false positive” rate is defined as the proportion of those without the disease who test positive. While a particular diagnostic method may not provide a definitive diagnosis of a condition, it suffices if the method provides a positive indication that aids in diagnosis.

As used herein the phrase “diagnosing” refers to classifying a disease or a symptom, determining a severity of the disease, monitoring disease progression, forecasting an outcome of a disease and/or prospects of recovery. The term “detecting” may also optionally encompass any of the above. Diagnosis of a disease according to the present invention can be effected by determining a level of a polynucleotide or a polypeptide of the present invention in a biological sample obtained from the subject, wherein the level determined can be correlated with predisposition to, or presence or absence of the disease. It should be noted that a “biological sample obtained from the subject” may also optionally comprise a sample that has not been physically removed from the subject, as described in greater detail below.

As defined herein, a therapeutically effective amount of a compound (I.e., an effective dosage) means an amount sufficient to produce a therapeutically (e.g., clinically) desirable result. The compositions can be administered one from one or more times per day to one or more times per week; including once every other day. The skilled artisan will appreciate that certain factors can influence the dosage and timing required to effectively treat a subject, including but not limited to the severity of the disease or disorder, previous treatments, the general health and/or age of the subject, and other diseases present. Moreover, treatment of a subject with a therapeutically effective amount of the compounds of the invention can include a single treatment or a series of treatments.

The term “sample” is meant to be interpreted in its broadest sense. A “sample” refers to a biological sample, such as, for example; one or more cells, tissues, or fluids (including, without limitation, plasma, serum, whole blood, cerebrospinal fluid, lymph, tears, urine, saliva, milk, pus, and tissue exudates and secretions) isolated from an individual or from cell culture constituents, as well as samples obtained from, for example, a laboratory procedure. A biological sample may comprise chromosomes isolated from cells (e.g., a spread of metaphase chromosomes), organelles or membranes isolated from cells, whole cells or tissues, nucleic acid such as genomic DNA in solution or bound to a solid support such as for Southern analysis, RNA in solution or bound to a solid support such as for Northern analysis, cDNA in solution or bound to a solid support, oligonucleotides in solution or bound to a solid support, polypeptides or peptides in solution or bound to a solid support, a tissue, a tissue print and the like.

Numerous well known tissue or fluid collection methods can be utilized to collect the biological sample from the subject in order to determine the level of DNA, RNA and/or polypeptide of the variant of interest in the subject. Examples include, but are not limited to, fine needle biopsy, needle biopsy, core needle biopsy and surgical biopsy (e.g., brain biopsy), and lavage. Regardless of the procedure employed, once a biopsy/sample is obtained the level of the variant can be determined and a diagnosis can thus be made.

### Compositions

Proteinuria can be primarily caused by alterations of structural proteins involved in the cellular mechanism of filtration. The pathophysiological causes of proteinuria can be divided in the following major groups: (1) genetically determined disturbances of the structures which form the “glomerular filtration unit” like the glomerular basement membrane, the podocytes, or the slit diaphragm, (2) inflammatory processes, either directly caused by autoimmune processes or indirectly induced by microbes, (3) damage of the glomeruli caused by agents, or (4) as the final result of progressive tubulointerstitial injury finally resulting in the loss of function of the entire nephron.

The central metabolism of a cell can determine its short- and long-term structure and function. When a disease state arises, the metabolism (i.e., the transportation of nutrients into the cells, the overall substrate utilization and production, synthesis and accumulation of intracellular metabolites, etc.) is altered in a way that may permit the cell to adapt under the changing physiologic constraints. Diabetes mellitus is a metabolic disease that also affects podocytes, key cells that regulate glomerular filtration. A pathological role for a cytoplasmic variant of cathepsin L enzyme as the biological instigator of kidney filter dysfunction (proteinuria) and progression of renal disease through cleavage of different types of critical podocyte target proteins. Podocytes are highly differentiated cells that reside in the kidney glomeruli. Their foot processes (FP) and interposed slit diaphragm (SD) form the final barrier to protein loss. Podocyte injury is typically associated with FP effacement and urinary protein loss.

In a healthy person, urinary protein excretion is less than 150 mg/day and consists mainly of filtered plasma proteins (60%) and tubular Tamm-Horsfall proteins (40%). The main plasma protein in the urine is albumin, constituting about 20% of daily protein excretion. In healthy subjects, the daily amount of urinary albumin is less than 20 mg (13.8 mg/min). Proteinuria usually reflects an increase in glomerular permeability for albumin and other plasma macromolecules. A 24-h urine collection containing more than 150 mg of protein is considered pathological. There are several basic types of proteinuria; for example, glomerular, tubular, overflow, and exercise-induced. Glomerular proteinuria is the most common form (around 90%). Low molecular weight molecules, such as β2-microglobulin, amino acids, and immunoglobulin light chains, have a molecular weight of about 25 kDa (albumin is 69 kDa). These smaller proteins are readily filtered across the glomerular filtration barrier and then fully reabsorbed by the proximal tubule. A variety of diseases that affect tubular and interstitial cell integrity impair the tubular reabsorption of these molecules. Some forms of glomerular diseases are also accompanied by tubular injury and tubular proteinuria.

Pathological processes, such as multiple myeloma with a production of paraproteins, can result in increased excretion of low molecular weight proteins into the urine, a process termed overflow proteinuria. In this scenario, proteinuria results from the amount of filtered proteins exceeding the reabsorptive capacity of the proximal tubule. Dynamic exercise can also result in increased urinary excretion of proteins, predominantly of plasma origin, during and following physical exercise. A number of terms have been used to describe this phenomenon—post-exercise proteinuria, athletic pseudonephritis, exercise proteinuria, or exercise-induced proteinuria. Maximal rates of proteinuria occur approximately 30 min after exercise, with a resolution toward resting levels within 24-48 h. The magnitude of proteinuria varies from near normal to heavy (47 g/day), with the greatest levels up to 100 times that of rest observed after high-intensity exercise, such as a marathon. It is noteworthy that post-exercise proteinuria is transient in nature and not associated with any particular renal disease, raising the intriguing possibility that at least some forms of proteinuria (e.g., post-exercise, post-prandial, infection-associated) may reflect a normal, physiological response of the human body. The work described herein, inter alia, proteinuria can result from enzymatic cleavage of essential regulators of podocyte actin dynamics by cytosolic cathepsin L (CatL).

Phosphorylation of synaptopodin by PKA or CaMKII promotes 14-3-3 binding, which protects synaptopodin against CatL-mediated cleavage, thereby stabilizing synaptopodin steady-state levels. Synaptopodin suppresses IRSp53:Mena-mediated filopodia by blocking the binding of Cdc42 and Mena to IRSp53 and induces stress fibers by competitive blocking the Smurf-1-mediated ubiquitination of RhoA. Synaptopodin also prevents the CatL-mediated degradation of dynamin. Synaptopodin stabilizes the kidney filter by blocking the re-organization of the podocyte actin cytoskeleton into a migratory phenotype. Dephosphorylation of synaptopodin by calcineurin abrogates the interaction with 14-3-3. This renders the CatL cleavage sites of synaptopodin accessible and promotes the degradation of synaptopodin. LPS or various other proximal signals induce the expression of B7-1 and CatL in podocytes, which cause proteinuria through the increased degradation of synaptopodin and dynamin. In parallel, LPS or other proximal signals can also activate Cdc42 and Rac 1 though uPAR:b3 integrin signaling, through the loss of synaptopodin-mediated inhibition of Cdc42 signaling or through Nef:Src-mediated activation of Rac1. As a consequence, the podocyte actin cytoskeleton shifts from a stationary to a motile phenotype, thereby causing foot process effacement and proteinuria. CsA and E64 safeguard against proteinuria by stabilizing synaptopodin and dynamin steady-state protein levels in podocytes, FP(4)-Mito by blocking Cdcd42:IRSp53:Mena signaling, cycloRGDfV by blocking uPAR:b3 integrin signaling, NSC23766 by blocking Rac1 and Epleronone by blocking aldosterone signaling.

The enzymatic regulation of CD2AP in podocytes was characterized herein, and cathepsin L mediated remodeling of CD2AP as responsible event for the progression of renal disease towards end-stage renal failure were identified. CD2AP is a scaffolding protein containing three N-terminal SH3 domains. In the kidney, it is strongly expressed in glomerular podocytes, cells that regulate renal filtration. Homozygous CD2AP mutation or haplo-insufficiency of the human CD2AP gene confer susceptibility to glomerular disease and mice lacking CD2AP develop progressive kidney failure. The structural organization of CD2AP at 21 Å resolution reveals a tetrameric structure that exposes two cathepsin L cleavage sites. CD2AP is processed into a 32 kD C-terminal, structurally competent core protein that lacks SH3 domains and permits the release of the slit diaphragm protein dendrin, that in turn translocates to the podocyte nucleus to promote podocyte apoptosis. Enzymatic remodeling of CD2AP by cytosolic cathepsin L occurs in human and murine progressive kidney disease. Cathepsin L knockout mice with serum nephritis and wild type mice expressing cleaving resistant CD2AP are protected from nuclear dendrin and glomerular disease progression. The data herein show that the proteolytic regulation of CD2AP constitutes a critical factor for renal disease progression.

Thus, in a preferred embodiment, a composition modulates expression and/or activity of cathepsin L. The agent can be any agent that modulates expression of cathepsin L or the activity of cathepsin L, such as for example, antisense oligonucleotides, antibodies, small molecules, and the like.

In an other preferred embodiment, an agent modulates the degradation of CD2AP. The agent can be an antibody, for example, which inhibits access of cathepsin and any other enzyme involved in the degradation of CD2AP to their specific cleavage sites. Thus, a composition may comprise both agents which inhibit cathepsin L expression and/or activity and an agent which inhibits CD2AP degradation.

In another preferred embodiment, an agent comprises a mutant CD2AP molecule which is resistant to cathepsin L enzymatic degradation. The examples which follow identify cathepsin L cleavage sites present in CD2AP. For example, amino acid sequences susceptible to cathepsin L activity comprise: ELRKE (SEQ ID NO: 1), ELAKA (SEQ ID NO: 2), LPGRF (SEQ ID NO: 3), AFVAR (SEQ ID NO: 4), LSAAE (SEQ ID NO: 5), ELGKE (SEQ ID NO: 6), QPLGS (SEQ ID NO: 7), KIRGI (SEQ ID NO: 8), APGSV (SEQ ID NO: 9), LIVGV (SEQ ID NO: 10), EIIRV (SEQ ID NO: 11), mutants, derivatives, variants or combinations thereof.

In a preferred embodiment, a blocking agent specific for one or more of these sites inhibit degradation of CD2AP by inhibiting access of the cathepsin enzyme.

In another preferred embodiment, a mutant CD2AP molecule comprises at least one nucleic acid or amino acid mutation in the enzyme cleavage sites.

In a preferred embodiment, the agent modulates or inhibits cathepsin L activity by about 5% as compared to a normal control, preferably by about 10%, preferably by about 50%, preferably by about 80%, 90%, 100%. Modulation of the activity of cathepsin L and stabilizes potential cleavage targets of the enzyme, thus protecting podocyte function and treating proteinuria.

In another preferred embodiment, the agent modulates or inhibits the degradation and/or rate of degradation of CD2AP molecules as compared to normal controls by about 5%, preferably by about 50%, preferably by about 80%, 90%, 100%.

In another preferred embodiment, agents which modulate cathepsin-L activity and/or expression comprise oligonucleotides, polynucleotides, peptides, polypeptides, antibodies, aptamers, small molecules, organic molecules, inorganic molecules or combinations thereof.

In another preferred embodiment, the composition comprises one or more agents which modulate CD2AP degradation or rate of degradation and/or cathepsin L activity, function or expression. For example, one agent directly inhibits cathepsin L activity. In another example, an agent directly inhibits CD2AP degradation and/or rate of degradation and a second agent which directly targets cathepsin L, by, for example, binding to it, such as an antibody, an antisense oligonucleotide which inhibits cathepsin L expression, an agent which targets another molecule in the cathepsin L synthesis pathway, or molecules in pathways which are targeted by cathepsin L, such as for example, dynamin, CD2AP, synaptopodin, etc. In another example, a composition comprises two agents whereby both modulate CD2AP degradation.

In another preferred embodiment, a method of treating a disease or disorder associated with pathological cathepsin L expression and/or activity comprises administering to a patient in need thereof, an effective amount of an agent which modulates cathepsin L activity, function and/or expression in vivo for treating the disorders. For example a podocyte disease or disorder such as proteinuria.

In another preferred embodiment, a method of treating a disease or disorder associated with pathological CD2AP degradation comprises administering to a patient in need thereof, an effective amount of an agent which modulates CD2AP degradation in vivo for treating the disorders.

In another preferred embodiment, a method of treating a disease or disorder associated with pathological CD2AP degradation comprises administering to a patient in need thereof, an effective amount of an agent which modulates CD2AP expression, activity and/or function in vivo for treating the disorders. For example, the agent can be a vector expression CD2AP molecules, an agent which targets CD2AP nucleic acids which increase in vivo production of CD2AP, a vector expressing a mutant form of CD2AP which is resistant to cleavage by cathepsin and other enzymes and the like.

In another preferred embodiment, a combination of agents which modulate CD2AP expression, function and/or activity and/or modulate CD2AP degradation are administered to a patient, for example, in the treatment of a disease or disorder characterized by proteinuria and/or podocyte diseases or disorders.

In a preferred embodiment, a disease or disorder characterized by proteinuria comprising: glomerular diseases, membranous glomerulonephritis, focal segmental glomerulonephritis, minimal change disease, nephrotic syndromes, pre-eclampsia, eclampsia, kidney lesions, collagen vascular diseases, stress, strenuous exercise, benign orthostatic (postural) proteinuria, focal segmental glomerulosclerosis (FSGS), IgA nephropathy, IgM nephropathy, membranoproliferative glomerulonephritis, membranous nephropathy, sarcoidosis, Alport's syndrome, diabetes mellitus, kidney damage due to drugs, Fabry's disease, infections, aminoaciduria, Fanconi syndrome, hypertensive nephrosclerosis, interstitial nephritis, Sickle cell disease, hemoglobinuria, multiple myeloma, myoglobinuria, cancer, Wegener's Granulomatosis or Glycogen Storage Disease Type 1.

In another preferred embodiment, modulation of CD2AP expression, function, activity, or degradation is modulated by an agent in the treatment of podocyte-related disorders or diseases. For the purposes of this invention, the terms “podocyte disease(s)” and “podocyte disorder(s)” are interchangeable and mean any disease, disorder, syndrome, anomaly, pathology, or abnormal condition of the podocytes or of the structure or function of their constituent parts.

In another preferred embodiment, a method of treating a podocyte disease or disorder associated with pathological cathepsin L expression and/or activity comprises administering to a patient in need thereof, an effective amount of an agent which modulates cathepsin L activity, function and/or expression in vivo for treating the podocyte diseases or disorders.

Such disorders or diseases include but are not limited to loss of podocytes (podocytopenia), podocyte mutation, an increase in foot process width, or a decrease in slit diaphragm length. In one aspect, the podocyte-related disease or disorder can be effacement or a diminution of podocyte density. In one aspect, the diminution of podocyte density could be due to a decrease in a podocyte number, for example, due to apoptosis, detachment, lack of proliferation, DNA damage or hypertrophy.

In one embodiment, the podocyte-related disease or disorder can be due to a podocyte injury. In one aspect, the podocyte injury can be due to mechanical stress such as high blood pressure, hypertension, or ischemia, lack of oxygen supply, a toxic substance, an endocrinologic disorder, an infection, a contrast agent, a mechanical trauma, a cytotoxic agent (cis-platinum, adriamycin, puromycin), calcineurin inhibitors, an inflammation (e.g., due to an infection, a trauma, anoxia, obstruction, or ischemia), radiation, an infection (e.g., bacterial, fungal, or viral), a dysfunction of the immune system (e.g., an autoimmune disease, a systemic disease, or IgA nephropathy), a genetic disorder, a medication (e.g., anti-bacterial agent, anti-viral agent, anti-fungal agent, immunosuppressive agent, anti-inflammatory agent, analgesic or anticancer agent), an organ failure, an organ transplantation, or uropathy. In one aspect, ischemia can be sickle-cell anemia, thrombosis, transplantation, obstruction, shock or blood loss. In on aspect, the genetic disorders may include congenital nephritic syndrome of the Finnish type, the fetal membranous nephropathy or mutations in podocyte-specific proteins, such as α-actin-4, podocin and TRPC6.

In one aspect, the podocyte-related disease or disorder can be an abnormal expression or function of slit diaphragm proteins such as podocin, nephrin, CD2AP, cell membrane proteins such as TRPC6, and proteins involved in organization of the cytoskeleton such as synaptopodin, actin binding proteins, lamb-families and collagens. In another aspect, the podocyte-related disease or disorder can be related to a disturbance of the GBM, to a disturbance of the mesangial cell function, and to deposition of antigen-antibody complexes and anti-podocyte antibodies. In another aspect, the podocyte-related disease or disorder can be tubular atrophy.

In a preferred embodiment, the podocyte-related disease or disorder comprises proteinuria, such as microalbumiuria or macroalbumiuria. Thus, in some preferred embodiments, one or more agents which modulate CD2AP expression, function, activity, degradation, rate of degradation and/or inhibiting expression or activity of cathepsin L can be combined with one or more other chemotherapeutic compounds which are used to treat any of the podocyte diseases or disorders.

Constituents of the Kidney Filtration Barrier: The kidney glomerulus is a highly specialized vascular bed that ensures the selective ultrafiltration of plasma so that the essential proteins are retained in the blood. The glomerular basement membrane (GBM) provides the primary structural support for the glomerular tuft. The basic unit of the glomerular tuft is a single capillary. The fenestrated glomerular endothelial cells and mesangial cells are located inside the GBM, whereas podocytes are attached to the outer aspect of the GBM. The glomerular capillaries function as the filtration barrier. The filtration barrier is characterized by distinct charge and size selectivity, thereby ensuring that albumin and other plasma proteins are retained in the circulation. Proteinuria occurs when the permeability of the glomerular barrier is increased. Human monogenetic studies show that mutations affecting podocyte proteins, including α-actinin-4, CD2AP, nephrin, PLCE1, podocin, and TRPC6, lead to renal disease owing to disruption of the filtration barrier and rearrangement of the podocyte actin cytoskeleton. Additional proteins regulating the podocyte actin cytoskeleton, such as Rho GDIa, podocalyxin, FAT1, 22 Nck1/2 and synaptopodin, are also of importance for sustained function of the glomerular filtration barrier. The glomerular filter is the primary barrier for albumin and that the glomerular sieving coefficient for albumin is extremely low.

Podocytes are Pericyte-Like Cells with an Actin-Based Contractile Apparatus: Differentiated podocytes are mesenchymal-like cells that arise from epithelial precursors during renal development. Similar to pericytes, podocytes never embrace a capillary in total.10 Podocytes consist of three morphologically and functionally different segments: a cell body, major processes, and foot processes (FPs). From the cell body, major processes arise that split into FP. FPs contain an actin-based cytoskeleton that is linked to the GBM. Podocyte FPs form a highly branched interdigitating network with FPs of neighboring podocytes connected by the slit diaphragm (SD). The SD is a modified adherens junction that covers the 30-50 nm wide filtration slits, thereby establishing the final barrier to urinary protein loss. The extracellular portion of the SD is made up of rod-like units that are connected in the center to a linear bar, forming a zipper-like pattern, with pores about the same size as or smaller than albumin. The function of podocytes is largely based on their complex cell architecture, in particular on the maintenance of the normal FP structure with their highly ordered parallel contractile actin filament bundles. FPs are functionally defined by three membrane domains: the apical membrane domain, the SD, and the basal membrane domain or sole plate that is associated with the GBM. All three domains are physically and functionally linked to the FP actin cytoskeleton. Proteins regulating the plasticity of the podocyte actin cytoskeleton are therefore of critical importance for sustained function of the glomerular filter.

Signal Transduction at the SD Regulates Podocyte Actin Dynamics: At the SD, multiple membrane proteins are present that are connected to the actin cytoskeleton through a variety of adaptor and effector proteins that may function as a key sensor and regulator of the permanent changes in FP shape and length. Changes in podocyte FP dynamics need to be precisely coordinated with FPs of neighboring podocytes, thereby preserving the integrity of the filtration barrier during FP movements, with functional coupling of opposing FPs and signaling cascades on both sides of the SD. Mutations in the NPHS1 gene encoding for the SD protein nephrin have been identified as the cause of congenital nephrotic syndrome of the Finnish type. It is noteworthy that nephrin is connected to the actin cytoskeleton through several adapter proteins and has a pivotal part in the regulation of podocyte actin dynamics. A signaling pathway couples nephrin to the actin cytoskeleton through the adaptor protein Nck. After nephrin phosphorylation by Fyn, Nck binds to phospho-nephrin and Nck binds to N-WASP. This in turn leads to the activation of the Arp2/3 complex, a major regulator of actin dynamics.

Podocyte Dysfunction is the Common Thread in Proteinuric Diseases: Podocytes can be injured in many forms of human and experimental glomerular disease, including minimal change disease (MCD), focal segmental glomerulosclerosis (FSGS), membranous glomerulopathy, diabetic nephropathy, and lupus nephritis. Characteristic changes are actin cytoskeleton reorganization of the involved FP, which typically leads to FP effacement and SD disruption. Interference with any of the three FP domains changes the actin cytoskeleton from parallel contractile bundles into a dense network with FP effacement (reflected by the simplification of the FP structure and loss of the normal interdigitating pattern) and proteinuria. Causes of FP effacement and proteinuria include the following: (i) changes in SD structure or function, (ii) interference with the GBM or the podocyte-GBM interaction, (iii) dysfunction of the podocyte actin cytoskeleton, (iv) modulation of the negative surface charge of podocytes, and (v) activation of CatL-mediated proteolysis (see below).

In addition, disturbances in the transcriptional regulation of podocyte function, modulation of vascular endothelial growth factor, transforming growth factor-13, adiponectin, notch, or aPKClambda signaling can also contribute to the pathogenesis of FP effacement and proteinuria. The early structural changes in podocyte morphology, such as FP effacement and SD disruption, are fully reversible. From a clinical point of view it is important to recognize that persistent podocyte injury harbors great risk to severe and progressive glomerular damage. The persistence of podocyte injury can cause podocyte cell death (through apoptosis or necrosis) or podocyte detachment from the GBM. Through a series of ensuing changes that have been reviewed in detail elsewhere, the loss of podocyte ultimately leads to glomerulosclerosis and end-stage renal failure. Patients with MCD or membranous glomerulopathy can present over years with nephrotic-range proteinuria without progressing to end-stage renal failure. Thus, the role of proteinuria in the progression of kidney failure probably depends on the type and the route of protein loss; that is, protein loss across the filtration barrier versus misdirected filtration into the periglomerular interstitium.

Increased FP Motility and the Onset of Proteinuria: The podocyte FP actin cytoskeleton is highly dynamic, although the underlying mechanisms remained ill defined. Testaments to a dynamic FP regulation are experiments that used perfusion of rat kidneys with the polycation protamine sulfate (PS). This treatment causes FP effacement and SD disruption within 15 min and tyrosine phosphorylation of nephrin. The reperfusion with heparin for another 15 min can reverse PS-induced FP effacement and nephrin phosphorylation. PS-induced FP effacement involves the active reorganization of actin filaments, and disruption of the actin cytoskeleton by cytochalasin can prevent PS-induced FP effacement.

The Role of the Cytosolic CatL and B7-1 in the Pathogenesis of Proteinuria: CatL is a member to the cathepsin family of cysteine proteases, which are involved primarily in protein breakdown in the lysosome. As shown herein, the onset of proteinuria represents a migratory event in podocyte FP that is caused by the activation of CatL. Subsequently, as shown herein, a cytoplasmic variant of CatL in podocytes is required for the development of proteinuria in mice through a mechanism that involves the cleavage of the large GTPase dynamin and synaptopodin. The clinical relevance of these findings was underscored by the observation that increased podocyte CatL expression was found in a variety of human proteinuric kidney diseases, including MCD, membranous glomerulopathy, FSGS, and diabetic nephropathy. Together these results support the notion that CatL-mediated proteolysis may have a key function in the development of many forms of proteinuria.

The lipopolysaccharide (LPS) model of proteinuria also helped identifying an unanticipated role for costimulatory molecule B7-1 in podocytes as an inducible modifier of glomerular permselectivity. It is noteworthy that the expression of B7-1 in podocytes is correlated with the severity of human lupus nephritis, and mice lacking B7-1 are protected from LPS-induced proteinuria, suggesting a functional link between podocyte B7-1 expression and proteinuria. Functionally, LPS signaling through Toll-like receptor-4 reorganized the actin cytoskeleton of cultured podocytes. These findings also suggest a function for B7-1 in danger signaling by podocytes. LPS causes proteinuria by selectively targeting podocytes because podocyte-specific overexpression of CatL-resistant dynamin or synaptopodin is sufficient to safeguard against proteinuria. Key effectors of the LPS-induced proteinuria have been detected in podocytes in vivo in animals and in biopsies from patients with proteinuric kidneys diseases, including B7-1, CatL,60 and urokinase plasminogen activator receptor (uPAR). Although there is no report about cytosolic variant of cathepsin L in the proximal tubule, CatL is highly expressed in the tubular lysosomes.

Activation of Promigratmy Cdc42 and Rac1 in Podocytes Causes FP Effacement and Proteinuria: The Rho family of small GTPases (RhoA, Rac1, and Cdc42) controls signal-transduction pathways that influence many aspects of cell behavior, including actin dynamics. At the leading edge, Rac1 and Cdc42 promote cell motility through the formation of lamellipodia and filopodia, respectively. On the contrary, RhoA promotes the formation of contractile actin-myosin containing stress fibers in the cell body and at the rear.

Agents: A wide variety of agents can be used to target cathepsins, especially cathepsin L. These agents may be designed to target cathepsins by having an in vivo activity which reduces the expression and/or activity of cathepsin L. In some preferred embodiments, the agents target the calcineurin-CatL pathways, such as for example, the calcineurin-CatL pathway-dependent versus independent pathways, leading to proteinuria and/or progressive kidney disease. In some embodiments, the agents are novel calcineurin (synaptopodin) and CatL substrates (dynamin, synaptopodin), and/or inhibit cytosolic CatL. In embodiments, the agents are selective, antiproteinuric, and/or podocyte-protective drugs. In other embodiments, one or more agents are administered as part of a preventative or treatment regimen, either at the same time or at various times apart as determined by the attending medical practitioner.

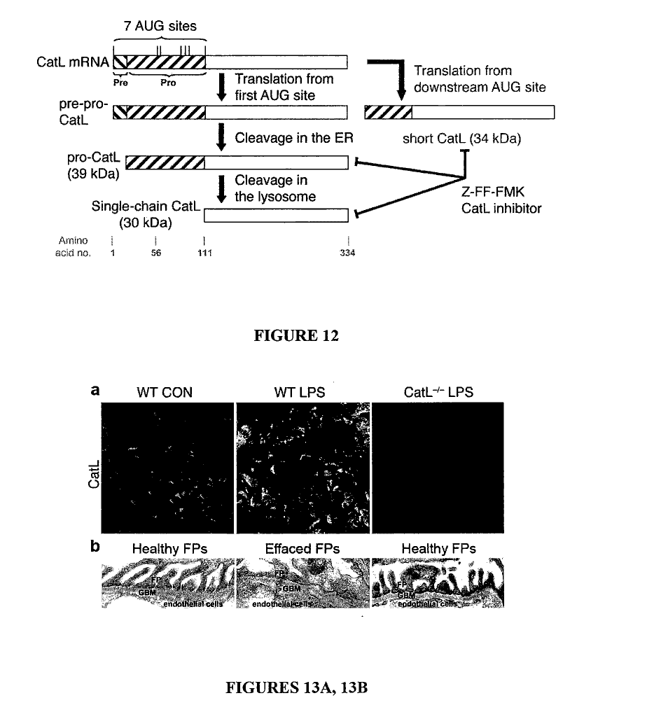

As shown in the examples section which follows, the role of podocyte cathepsin L is a key enzyme in acquired proteinuria. CatL is a potent endoprotease primarily responsible for final protein breakdown within lysosomal compartments. In addition, a secreted form of CatL is involved in the degradation of extracellular matrix (ECM) in vivo and in vitro. Both the lysosomal and secreted forms of CatL have been implicated in cancer cell biology and metastasis. A CatL inhibitor E64 can reduce experimental proteinuria in a rat glomerulonephritis model. The onset of experimental proteinuria is accompanied by an increased motility of podocytes, which was abrogated in CatL^{-/-} podocytes. Expression of a few intracellular podocyte proteins such as CD2AP declined, but only in the presence of CatL. In a subsequent study, we found that PAN and Lipopolysaccharide (LPS, another proteinuric stimulus) specifically induce a short cytosoplasmic variant of CatL devoid of the lysosomal targeting sequence (FIG. 12). A shorter CatL variant arises by translation from an alternate downstream AUG site and locates in the nucleus of fibroblasts where it can cleave the transcription factor CDP/Cux or serve in Histone H3 processing during mouse embryonic stem cell differentiation. This obviously broke with a dogma that CatL can only be active in the acidic pH of the lysosome. Whereas conventional CatL cleaves a variety of proteins very efficiently due to the denaturing conditions and low pH of the lysosome, short CatL exhibits a remarkable substrate specificity that allows a very specific enzymatic activity at cytosolic or nuclear pH. So far, two substrates of cytosolic CatL have been described in podocytes: dynamin and synaptopodin. Both proteins contribute to the functional F-actin in normal podocyte FPs and allow their effacement after their enzymatic processing by CatL.

CatL is significantly induced in at least two rodent models of proteinuria, i.e. the LPS mouse model (FIG. 13A) and the rat PAN model. Stainings in cultured podocytes treated with LPS or PAN revealed a vast increase of CatL enzyme in the cytosol. Enzymatic activity assays determined that cytosolic CatL is enzymatically active and can cleave its targets dynamin and synaptopodin. The significance of CatL induction is further underscored by the finding that CatL knockout mice are protected from LPS induced FP effacement and proteinuria (FIGS. 13A, 13B). Human data stems from isolated glomeruli of explanted renal allografts with chronic allograft nephropathy and microdissected glomeruli from kidney biopsies of patients with three types of glomerular disease, membranous nephropathy (MN), focal segmental glomerulosclerosis (FSGS) and diabetic nephropathy (DNP) (FIG. 14A). All these cases revealed a two-fold or greater induction of CatL mRNA as measured by real-time RT-PCR. Increased CatL protein is found in podocytes of patients with DNP [15] (FIG. 14B, 14C).

Cathepsin L proteolyzes dynamin and synaptopodin: The computer algorithm PEPS (Prediction of Endopeptidase Proteolytic Sites) has served to identify possible CatL substrates. Since PEPS does not take into account the condition of the environment, i.e. the pH of the compartment (lysososome vs cytosol), it is necessary to experimentally confirm the cleavage prediction using purified proteins. Using this algorithm, the first identified cleavage target in podocytes was the large GTPase dynamin. Dynamin is essential for the formation of clathrin-coated vesicles at the plasma membrane during endocytosis and has also been implicated in the regulation of actin dynamics in certain cell types. Dynamin is specifically cleaved in podocytes by CatL during LPS- or PAN-induced proteinuria in animal models and gene delivery of mutant dynamin forms resistant to cleavage by CatL protected mice from LPS-induced proteinuria. Intact dynamin is required for proper podocyte structure and function. Expression of dominant-negative dynamin mutants in podocytes caused proteinuria in vivo and led to a loss of actin stress fibers in vitro. The role of dynamin in maintaining podocyte integrity does not depend on its function in endocytosis, but rather on its ability to stabilize F-actin organization in the FPs.

Synaptopodin is another major cleavage target for cytoplasmic CatL. Synaptopodin is the founding member of a unique class of proline-rich, actin-associated proteins that are expressed in highly dynamic cell compartements, such as the dendritic spine apparatus of neurons and podocyte FPs. Synaptopodin binds to α-actinin and regulates the actin-bundling activity of α-actinin. Synaptopodin-deficient (synpo^{-/-}) mice display impaired recovery from protamine sulfate-induced podocyte FP effacement and LPS-induced proteinuria. Similarly, synpo^{-/-} podocytes show impaired actin filament reformation in vitro. Synaptopodin is specifically proteolyzed at two cleavage sites by cytosolic CatL. In vivo gene delivery or the podocyte-specific transgenic expression of a synaptopodin mutant that lacks these cleavage sites protected mice from LPS-induced proteinuria, suggesting that CatL-mediated cleavage of synaptopodin is required for the induction of FP effacement by LPS. Stabilized synaptopodin protein levels also help to maintain dynamin levels.

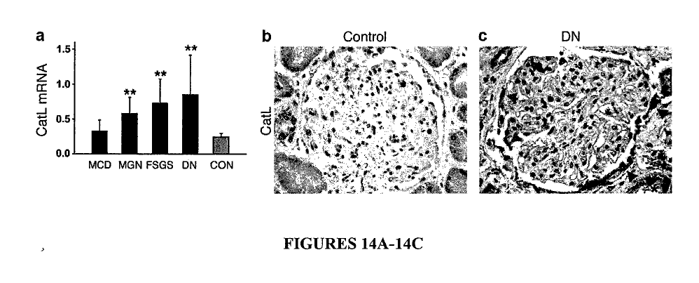

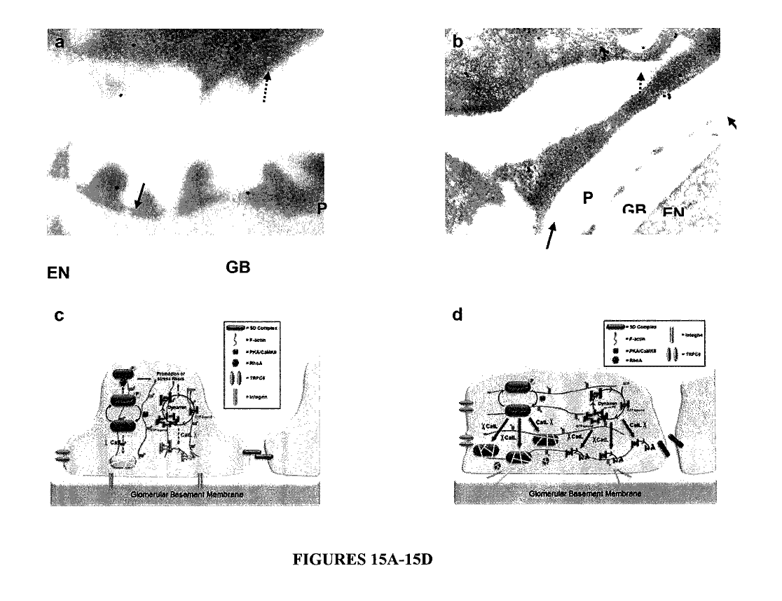

The main deleterious action of CatL in podocytes stems from a novel CatL form that is active in the cytoplasm of podocytes (FIG. 15A-15D) and that is highly target selective. Embodiments of the invention are also directed to CatL inhibitors that localize to the cytosol of a podocyte and specifically inhibit the disease-causing CatL variant.

In summary, podocyte FP effacement can be caused by the translation of a novel CatL variant in the cytosol of podocyte FPs. CatL is induced in many proteinuric diseases. So far two major cleavage targets have been described: Dynamin and synaptopodin. Both proteins are regulators of podocyte cytoskeletal function. Additional targets are being investigated. The unraveling of these pathways not only greatly enhances our understanding of the pathophysiology of glomerular diseases but also enables the development of specific therapies for proteinuric syndromes by directly targeting components of these enzymatic cascades in podocytes.

In other preferred embodiments, the agents may regulate cathepsin L based on the cDNA or regulatory regions of cathepsin L. For example, DNA-based agents, such as antisense inhibitors and ribozymes, can be utilized to target both the introns and exons of the cathepsin genes as well as at the RNA level.

Alternatively, the agents may target cathepsin L based on the amino acid sequences including the propieces and/or three-dimensional protein structures of cathepsin L. Protein-based agents, such as human antibody, non-human monoclonal antibody and humanized antibody, can be used to specifically target different epitopes on cathepsin L. Peptides or peptidomimetics can serve as high affinity inhibitors to specifically bind to the active site of a particular cathepsin, thereby inhibiting the in vivo activity of the cathepsin. Small molecules may also be employed to target cathepsin, especially those having high selectivity toward cathepsin L.

In addition to targeting cathepsin L, agents may also be used which competitively inhibit cathepsin L by competing with the natural substrates of cathepsins for binding with the enzymes.

In another embodiment, one of the agents can be a are protease inhibitor, specific for cathepsin L. Inhibitors of cathepsins include cathepsin L, B, and D inhibitors, antisense to cathepsin, siRNA, and antisense-peptide sequences. Examples of cathepsin inhibitors include but are not limited to epoxysuccinyl peptide derivatives [E-64, E-64a, E-64b, E-64c, E-64d, CA-074, CA-074 Me, CA-030, CA-028, etc.], peptidyl aldehyde derivatives [leupeptin, antipain, chymostatin, Ac-LVK-CHO₅ Z-Phe-Tyr-CHO, Z-Phe-Tyr(OtBu)-COCHO.H₂0, 1-Naphthalenesulfonyl-Ile-Trp-CHO, Z-Phe-Leu-COCHO.H₂O, etc.], peptidyl semicarbazone derivatives, peptidyl methylketone derivatives, peptidyl trifluoromethylketone derivatives [Biotin-Phe-Ala-fluoromethyl ketone, Z-Leu-Leu-Leu-fluoromethyl ketone minimum, Z-Phe-Phe-fluoromethyl ketone, N-Methoxysuccinyl-Phe-HOMO-Phe-fluoromethyl ketone, Z-Leu-Leu-Tyr-fluoromethyl ketone, Leupeptin trifluoroacetate, ketone, etc.], peptidyl halomethylketone derivatives [TLCK, etc.], bis(acylamino)ketone [1,3-Bis(CBZ-Leu-NH)-2-propanone, etc.], peptidyl diazomethanes [Z-Phe-Ala-CHN₂, Z-Phe-Thr(OBzl)-CHN₂, Z-Phe-Tyr (O-t-But)-CHN₂, Z-Leu-Leu-Tyr-CHN₂, etc.], peptidyl acyloxymethyl ketones, peptidyl methylsulfonium salts, peptidyl vinyl sulfones [LHVS, etc.], peptidyl nitriles, disulfides [5,5′-dithiobis[2-nitrobenzoic acid], cysteamines, 2,2′-dipyridyl disulfide, etc.], non-covalent inhibitors [N-(4-Biphenylacetyl)-S-methylcysteine-(D)-Arg-Phe-b-phenethylamide, etc.], thiol alkylating agents [maleimides, etc,], azapeptides, azobenzenes, O-acylhydroxamates [Z-Phe-Gly-NHO-Bz, Z-FG-NHO-BzOME, etc.], lysosomotropic agents [chloroquine, ammonium chloride, etc.], and inhibitors based on Cystatins [Cystatins A, B, C, stefins, kininogens, Procathepsin B Fragment 26-50, Procathepsin B Fragment 36-50, etc.].

In another embodiment, the invention provides methods for inhibiting at least one enzymatic activity of cathepsin L. In one embodiment the cathepsin L inhibitors comprise: Z-Phe-Phe-FMK, H-Arg-Lys-Leu-Trp-NH₂, N-(I-Naphthalenylsulfonyl)-ile-Trp-aldehyde, Z-Phe-Tyr(tBu)-diazomethylketone, or Z-Phe-Tyr-aldehyde .

Nucleic Acid-based Agents: Nucleic acid-based agents such as antisense molecules and ribozymes can be utilized to target both the introns and exons of the cathepsin genes as well as at the RNA level to inhibit gene expression thereof, thereby inhibiting the activity of the targeted cathepsin. Further, triple helix molecules may also be utilized in inhibiting the cathepsin gene activity. Such molecules may be designed to reduce or inhibit either the wild type cathepsin gene, or if appropriate, the mutant cathepsin gene activity. Techniques for the production and use of such molecules are well known to those of skill in the art, and are succinctly described below.

In another preferred embodiment, CD2AP genes are modulated by targeting nucleic acid sequences involved in the expression and/or activity of CD2AP molecules. For example, regulatory regions would be a target to increase the expression of CD2AP.

Antisense RNA and DNA molecules act to directly block the translation of mRNA by hybridizing to targeted mRNA and preventing protein translation. Antisense approaches involve the design of oligonucleotides that are complementary to a target gene mRNA. The antisense oligonucleotides will bind to the complementary target gene mRNA transcripts and prevent translation. Absolute complementarity, although preferred, is not required.

A sequence “complementary” to a portion of an RNA, as referred to herein, means a sequence having sufficient complementarity to be able to hybridize with the RNA, forming a stable duplex; in the case of double-stranded antisense nucleic acids, a single strand of the duplex DNA may thus be tested, or triplex formation may be assayed. The ability to hybridize will depend on both the degree of complementarity and the length of the antisense nucleic acid. Generally, the longer the hybridizing nucleic acid, the more base mismatches with an RNA it may contain and still form a stable duplex (or triplex, as the case may be). One skilled in the art can ascertain a tolerable degree of mismatch by use of standard procedures to determine the melting point of the hybridized complex.

Oligonucleotides that are complementary to the 5′ end of the message, e.g., the 5′ untranslated sequence up to and including the AUG initiation codon, should work most efficiently at inhibiting translation. However, sequences complementary to the 3′ untranslated sequences of mRNAs have been shown to be effective at inhibiting translation of mRNAs as well. Wagner (1994) Nature 372:333-335. For example, oligonucleotides complementary to either the 5′- or 3′-untranslated, non-coding regions of the human or mouse gene of cathepsin L could be used in an antisense approach to inhibit translation of endogenous cathepsin L mRNA.

In another preferred embodiment, the antisense approach can be used to target negative regulators of CD2AP expression and/or function.

Oligonucleotides complementary to the 5′ untranslated region of the mRNA should include the complement of the AUG start codon. Antisense oligonucleotides complementary to mRNA coding regions are less efficient inhibitors of translation but could be used in accordance with the invention. Whether designed to hybridize to the 5′-, 3′- or coding region of target gene mRNA, antisense nucleic acids are preferably at least six nucleotides in length, and are more preferably oligonucleotides ranging from 6 to about 50 nucleotides in length. In specific aspects the oligonucleotide is at least 10 nucleotides, preferably at least 17 nucleotides, more preferably at least 25 nucleotides and most preferably at least 50 nucleotides.

Alternatively, antisense molecules may be designed to target the translated region, i.e., the cDNA of the cathepsin gene. For example, the antisense RNA molecules targeting the full coding sequence or a portion of the mature murine cathepsin L (Kirschke et al. (2000) Euro. J. Cancer 36:787-795) may be utilized to inhibit expression of cathepsin L and thus reduce the activity of its enzymatic activity. In addition, a full length or partial cathepsin L cDNA can be subcloned into a pcDNA-3 expression vector in reversed orientation and such a construct can be transfected into cells to produce antisense polyRNA to block endogenous transcripts of a cathepsin, such as cathepsin L, and thus inhibit the cathepsin's expression.

In vitro studies may be performed to quantitate the ability of the antisense oligonucleotide to inhibit gene expression. It is preferred that these studies utilize controls that distinguish between antisense gene inhibition and nonspecific biological effects of oligonucleotides. It is also preferred that these studies compare levels of the target RNA or protein with that of an internal control RNA or protein. Additionally, it is envisioned that results obtained using the antisense oligonucleotide are compared with those obtained using a control oligonucleotide. It is preferred that the control oligonucleotide is of approximately the same length as the test oligonucleotide and that the nucleotide sequence of the oligonucleotide differs from the antisense sequence no more than is necessary to prevent specific hybridization to the target sequence.

The oligonucleotides can be DNA or RNA or chimeric mixtures or derivatives or modified versions thereof, single-stranded or double-stranded. The oligonucleotide can be modified at the base moiety, sugar moiety, or phosphate backbone, for example, to improve stability of the molecule, hybridization, etc. The oligonucleotide may include other appended groups such as peptides, or agents facilitating transport across the cell membrane (See, e.g., Letsinger (1989) Proc. Natl. Acad. Sci. U.S.A. 86:6553-6556) or the blood-brain barrier, hybridization-triggered cleavage agents. See, e.g., Krol (1988) Bio Techniques 6:958-976 or intercalating agents. See, e.g., Zon (1988) Pharm. Res. 5:539-549. The oligonucleotide may be conjugated to another molecule, e.g., a peptide, hybridization triggered cross-linking agent, transport agent, hybridization-triggered cleavage agent, etc.

The antisense oligonucleotide may comprise at least one modified base moiety which is selected from the group consisting of, but not being limited to, 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomet-hyluracil, dihydrouracil, β-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, β-D-mannosylqueosine, 5′-methoxycarboxymethyluracil, 5-methoxyuracil, 2-methylthio-N-6-isopenten-yladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine.

The antisense oligonucleotide may also comprise at least one modified sugar moiety selected from the group consisting of, but not being limited to arabinose, 2-fluoroarabinose, xylulose, and hexose.

In yet another embodiment, the antisense oligonucleotide comprises at least one modified phosphate backbone selected from the group consisting of a phosphorothioate, a phosphorodithioate, a phosphoramidothioate, a phosphoramidate, a phosphordiamidate, a methylphosphonate, an alkyl phosphotriester, and a formacetal or analog thereof.

Ribozyme molecules designed to catalytically cleave target gene mRNA transcripts can also be used to prevent translation of target gene mRNA and, therefore, expression of target gene product. See, e.g. Sarver et al. (1990) Science 247:1222-1225.

Ribozymes are enzymatic RNA molecules capable of catalyzing the specific cleavage of RNA. The mechanism of ribozyme action involves sequence specific hybridization of the ribozyme molecule to complementary target RNA, followed by an endonucleolytic cleavage event. The composition of ribozyme molecules should include one or more sequences complementary to the target gene mRNA, and should include the well known catalytic sequence responsible for mRNA cleavage.

While ribozymes that cleave mRNA at site specific recognition sequences can be used to destroy target gene mRNAs, the use of hammerhead ribozymes is preferred. Hammerhead ribozymes cleave mRNAs at locations dictated by flanking regions that form complementary base pairs with the target mRNA. The sole requirement is that the target mRNA have the following sequence of two bases: 5′-UG-3′. The construction and production of hammerhead ribozymes is well known in the art.

Endogenous cathepsin gene expression can also be reduced by inactivating or “knocking out” the targeted cathepsin gene or its promoter using targeted homologous recombination. Smithies et al. (1985) Nature 317:230-234; Thomas and Capecchi, (1987) Cell 51:503-512; and Thompson et al. (1989) Cell 5:313-321.

Alternatively, endogenous cathepsin gene expression can be reduced by targeting deoxyribonucleotide sequences complementary to the regulatory region of the cathepsin gene (i.e., the target gene promoter and/or enhancers) to form triple helical structures that prevent transcription of the target gene in target cells in the body. See generally, Helene (1991) Anticancer Drug Des. 6:569-584; Helene et al. (1992) Ann. N.Y. Acad. Sci. 660:27-36; and Maher (1992) Bioassays 14:807-815.

Nucleic acid molecules to be used in triplex helix formation for the inhibition of transcription should be single stranded and composed of deoxynucleotides. The base composition of these oligonucleotides must be designed to promote triple helix formation via Hoogsteen base pairing rules, which generally require sizeable stretches of either purines or pyrimidines to be present on one strand of a duplex. Nucleotide sequences may be pyrimidine-based, which will result in TAT and CGC triplets across the three associated strands of the resulting triple helix. The pyrimidine-rich molecules provide base complementarity to a purine-rich region of a single strand of the duplex in a parallel orientation to that strand. In addition, nucleic acid molecules may be chosen that are purine-rich, for example, contain a stretch of G residues. These molecules will form a triple helix with a DNA duplex that is rich in GC pairs, in which the majority of the purine residues are located on a single strand of the targeted duplex, resulting in GGC triplets across the three strands in the triplex.

### Biomarkers

In a preferred embodiment, a biomarker for the diagnosis of a disease or disorder characterized by proteinuria and/or identification of individuals at risk of developing a disease or disorder characterized by proteinuria comprising: cathepsin-L, system N glutamine transporter (SNAT3), dynamin, synaptopodin or cytoskeletal regulator protein synaptopodin, cytoskeletal adaptor protein (CD2AP), variants, mutants or fragments thereof.

The biomarkers can be increased or decreased in expression relative to each other. The panel of biomarker expression profiles are compared to normal controls. In other instances, the intra-cellular localization changes with the progression of disease. For example, a fragment of CD2AP comprises p32 C-terminal fragment. As cathepsin-L cleaves the CD2AP, there is an increase in N-terminal CD2AP fragments and p32 fragments. The p32 cannot bind to dendrin, which is then trafficked to the podocyte nuclei. Thus, dendrin localization is altered during the disease progression.

In another preferred embodiment, the identification of an individual at risk of developing disease or disorder characterized by proteinuria detects at least one biomarker or fragments thereof.

In another preferred embodiment, the progression of disease or disorder characterized by proteinuria is correlated to an increase in cathepsin-L and/or system N glutamine transporter (SNAT3) expression and/or an increase in p32 CD2AP C-terminal fragment expression and/or dendrin in podocyte nuclei.

### Candidate Therapeutic Agents:

In a preferred embodiment, methods (also referred to herein as “screening assays”) are provided for identifying modulators, i.e., candidate or test compounds or agents (e.g., proteins, peptides, peptidomimetics, peptoids, small molecules, analogues or other drugs) which modulate CD2AP expression, function degradation and/or act directly on cathepsin L activity or expression or synthesis pathways thereof. Compounds thus identified can be used to modulate the activity of target gene products, prolong the half-life of a protein or peptide, regulate cell division, etc, in a therapeutic protocol, to elaborate the biological function of the target gene product, or to identify compounds that disrupt normal target gene interactions.

In another preferred embodiment, a high-throughput screening assay (HTS) screening assay is used to screen a diverse library of member compounds. The “compounds” or “candidate therapeutic agents” or “candidate agents” can be any organic, inorganic, small molecule, protein, antibody, aptamer, nucleic acid molecule, or synthetic compound.

In another preferred embodiment, the candidate agents modulate cathepsin enzymes, precursors or molecules involved in the pathways. Preferably, the enzyme is cathepsin L. These enzymes can be involved in various biochemical pathways such as synthetic pathways, breakdown pathways, e.g. ubiquitin, enzymatic pathways, protein trafficking pathways, metabolic pathways, signal transduction pathways, and the like.

In another preferred embodiment, the high throughput assays identifies candidate agents that target and modulate the pathways involved in the pathological expression or activity of cathepsin L The candidate agents would be useful in developing and identifying novel agents for the treatment of podocyte diseases or disorders, such as, for example, proteinuria.

In one embodiment, the invention provides assays for screening candidate or test compounds which modulate the degradation, rate of degradation, activity, expression and/or function of CD2AP. In some embodiments, an agent binds to CD2AP and inhibits cleavage or degradation of CD2AP.

In another embodiment, the invention provides assays for screening candidate or test compounds that bind to or modulate an activity of cathepsin L protein or polypeptide or a biologically active portion thereof, mutants or fragments, or fusion proteins thereof.

Candidate agents include numerous chemical classes, though typically they are organic compounds including small organic compounds, nucleic acids including oligonucleotides, and peptides. Small organic compounds suitably may have e.g. a molecular weight of more than about 40 or 50 yet less than about 2,500. Candidate agents may comprise functional chemical groups that interact with proteins and/or DNA.

The test compounds of the present invention can be obtained using any of the numerous approaches in combinatorial library methods known in the art, including: biological libraries; peptoid libraries (libraries of molecules having the functionalities of peptides, but with a novel, non-peptide backbone which are resistant to enzymatic degradation but which nevertheless remain bioactive; see, e.g., Zuckermann, R. N. et al. (1994) J. Med. Chem. 37:2678-85); spatially addressable parallel solid phase or solution phase libraries; synthetic library methods requiring deconvolution; the one-bead one-compound library method; and synthetic library methods using affinity chromatography selection. The biological library and peptoid library approaches are limited to peptide libraries, while the other four approaches are applicable to peptide, non-peptide oligomer or small molecule libraries of compounds (Lam (1997) Anticancer Drug Des. 12:145).

Examples of methods for the synthesis of molecular libraries can be found in the art, for example in: DeWitt et al. (1993) Proc. Natl. Acad. Sci. U.S.A. 90:6909; Erb et al. (1994) Proc. Natl. Acad. Sci. USA 91:11422; Zuckermann et al. (1994). J. Med. Chem. 37:2678; Cho et al. (1993) Science 261:1303; Carrell et al. (1994) Angew. Chem. Int. Ed. Engl. 33:2059; Carell et al. (1994) Angew. Chem. Int. Ed. Engl. 33:2061; and Gallop et al. (1994) J. Med. Chem. 37:1233.

Libraries of compounds may be presented in solution (e.g., Houghten (1992) Biotechniques 13:412-421), or on beads (Lam (1991) Nature 354:82-84), chips (Fodor (1993) Nature 364:555-556), bacteria (Ladner, U.S. Pat. No. 5,223,409), spores (Ladner U.S. Pat. No. 5,223,409), plasmids (Cull et al. (1992) Proc Nat'l Acad Sci USA 89:1865-1869) or on phage (Scott and Smith (1990) Science 249:386-390; Devlin (1990) Science 249:404-406; Cwirla et al. (1990) Proc. Natl. Acad. Sci. 87:6378-6382; Felici (1991) J. Mol. Biol. 222:301-310; Ladner supra.).

In another preferred embodiment, the candidate therapeutic agent comprises, proteins, peptides, organic molecules, inorganic molecules, nucleic acid molecules, and the like. These molecules can be natural, e.g. from plants, fungus, bacteria etc., or can be synthesized or synthetic.

A prototype compound may be believed to have therapeutic activity on the basis of any information available to the artisan. For example, a prototype compound may be believed to have therapeutic activity on the basis of information contained in the Physician's Desk Reference. In addition, by way of non-limiting example, a compound may be believed to have therapeutic activity on the basis of experience of a clinician, structure of the compound, structural activity relationship data, EC₅₀, assay data, IC₅₀ assay data, animal or clinical studies, or any other basis, or combination of such bases.

A therapeutically-active compound is a compound that has therapeutic activity, including for example, the ability of a compound to induce a specified response when administered to a subject or tested in vitro. Therapeutic activity includes treatment of a disease or condition, including both prophylactic and ameliorative treatment. Treatment of a disease or condition can include improvement of a disease or condition by any amount, including prevention, amelioration, and elimination of the disease or condition. Therapeutic activity may be conducted against any disease or condition, including in a preferred embodiment against any disease or disorder associated with proteinuria. In order to determine therapeutic activity any method by which therapeutic activity of a compound may be evaluated can be used. For example, both in vivo and in vitro methods can be used, including for example, clinical evaluation, EC₅₀, and IC₅₀ assays, and dose response curves.

Candidate compounds for use with an assay of the present invention or identified by assays of the present invention as useful pharmacological agents can be pharmacological agents already known in the art or variations thereof or can be compounds previously unknown to have any pharmacological activity. The candidate compounds can be naturally occurring or designed in the laboratory. Candidate compounds can comprise a single diastereomer, more than one diastereomer, or a single enantiomer, or more than one enantiomer.

Candidate compounds can be isolated, from microorganisms, animals or plants, for example, and can be produced recombinantly, or synthesized by chemical methods known in the art. If desired, candidate compounds of the present invention can be obtained using any of the numerous combinatorial library methods known in the art, including but not limited to, biological libraries, spatially addressable parallel solid phase or solution phase libraries, synthetic library methods requiring deconvolution, the “one-bead one-compound” library method, and synthetic library methods using affinity chromatography selection. The biological library approach is limited to polypeptide libraries. The other four approaches are applicable to polypeptide, non-peptide oligomer, or small molecule libraries of compounds and are preferred approaches in the present invention. See Lam, Anticancer Drug Des. 12: 145-167 (1997).

In an embodiment, the present invention provides a method of identifying a candidate compound as a suitable prodrug. A suitable prodrug includes any prodrug that may be identified by the methods of the present invention. Any method apparent to the artisan may be used to identify a candidate compound as a suitable prodrug.

In another aspect, the present invention provides methods of screening candidate compounds for suitability as therapeutic agents. Screening for suitability of therapeutic agents may include assessment of one, some or many criteria relating to the compound that may affect the ability of the compound as a therapeutic agent. Factors such as, for example, efficacy, safety, efficiency, retention, localization, tissue selectivity, degradation, or intracellular persistence may be considered. In an embodiment, a method of screening candidate compounds for suitability as therapeutic agents is provided, where the method comprises providing a candidate compound identified as a suitable prodrug, determining the therapeutic activity of the candidate compound, and determining the intracellular persistence of the candidate compound. Intracellular persistence can be measured by any technique apparent to the skilled artisan, such as for example by radioactive tracer, heavy isotope labeling, or LCMS.

In screening compounds for suitability as therapeutic agents, intracellular persistence of the candidate compound is evaluated. In a preferred embodiment, the agents are evaluated for their ability to modulate the intracellular pH may comprise, for example, evaluation of intracellular pH over a period of time in response to a candidate therapeutic agent. In a preferred embodiment, the intra-podocyte pH in the presence or absence of the candidate therapeutic compound in human tissue is determined. Any technique known to the art worker for determining intracellular pH may be used in the present invention. See, also, the experimental details in the examples section which follows.

A further aspect of the present invention relates to methods of inhibiting the activity of a condition or disease associated with proteinuria comprising the step of treating a sample or subject believed to have a disease or condition with a prodrug identified by a compound of the invention. Compositions of the invention act as identifiers for prodrugs that have therapeutic activity against a disease or condition. In a preferred aspect, compositions of the invention act as identifiers for drugs that show therapeutic activity against conditions including for example associated with proteinuria.

In one embodiment, a screening assay is a cell-based assay in which the activity of cathepsin L is measured against an increase or decrease of pH values in the cells. Determining the ability of the test compound to modulate the pH and determining cathepsin L activity, by various methods, including for example, fluorescence, protein assays, blots and the like. The cell, for example, can be of mammalian origin, e.g., human.

In another preferred embodiment, the screening assay is a high-throughput screening assay. The ability of a compound to modulate CD2AP degradation, expression, function etc., and/or modulate cathepsin L expression and/or activity can be evaluated as described in detail in the Examples which follow.

In another preferred embodiment, soluble and/or membrane-bound forms of isolated proteins, mutants or biologically active portions thereof, can be used in the assays if desired. When membrane-bound forms of the protein are used, it may be desirable to utilize a solubilizing agent. Examples of such solubilizing agents include non-ionic detergents such as n-octylglucoside, n-dodecylglucoside, n-dodecylmaltoside, octanoyl-N-methylglucamide, decanoyl-N-methylglucamide, TRITON™ X-100, TRITON™ X-114, THESIT™, Isotridecypoly(ethylene glycol ether)ₙ, 3-[(3-cholamidopropyl)dimethylamminio]-1-propane sulfonate (CHAPS), 3-[(3-cholamidopropyl)dimethylamminio]-2-hydroxy-1-propane sulfonate (CHAPSO), or N-dodecyl=N,N-dimethyl-3-ammonio-1-propane sulfonate.

Cell-free assays can also be used and involve preparing a reaction mixture which includes cathepsin L, CD2AP and the test compound under conditions and time periods to allow the measurement of the cathepsin L activity over time, CD2AP degradation rates, increases in CD2AP activity, etc, over a range of values and concentrations of test agents.

The enzymatic activity can be also be detected, e.g., using fluorescence energy transfer (FET) (see, for example, Lakowicz et al., U.S. Pat. No. 5,631,169; Stavrianopoulos, et al, U.S. Pat. No. 4,868,103). A fluorophore label on the first, ‘donor’ molecule is selected such that its emitted fluorescent energy will be absorbed by a fluorescent label on a second, ‘acceptor’ molecule, which in turn is able to fluoresce due to the absorbed energy. Alternately, the ‘donor’ protein molecule may simply utilize the natural fluorescent energy of tryptophan residues. Labels are chosen that emit different wavelengths of light, such that the ‘acceptor’ molecule label may be differentiated from that of the ‘donor’. Since the efficiency of energy transfer between the labels is related to the distance separating the molecules, the spatial relationship between the molecules can be assessed. In a situation in which binding occurs between the molecules, the fluorescent emission of the ‘acceptor’ molecule label in the assay should be maximal. A FET binding event can be conveniently measured through standard fluorometric detection means well known in the art (e.g., using a fluorimeter).

In another embodiment, determining the ability of the enzyme (e.g. cathepsin L) to bind or “dock” to its binding site on a target molecule (CD2AP) can be accomplished using real-time Biomolecular Interaction Analysis (BIA) (see, e.g., Sjolander, S. and Urbaniczky, C. (1991) Anal. Chem. 63:2338-2345 and Szabo et al. (1995) Curr. Opin. Struct. Biol. 5:699-705). “Surface plasmon resonance” or “BIA” detects biospecific interactions in real time, without labeling any of the interactants (e.g., BLAcore). Changes in the mass at the binding surface (indicative of a binding event) result in alterations of the refractive index of light near the surface (the optical phenomenon of surface plasmon resonance (SPR)), resulting in a detectable signal which can be used as an indication of real-time reactions between biological molecules.

In one embodiment, the target product or the test substance is anchored onto a solid phase. The target product/test compound complexes anchored on the solid phase can be detected at the end of the reaction. Preferably, the target product can be anchored onto a solid surface, and the test compound, (which is not anchored), can be labeled, either directly or indirectly, with detectable labels discussed herein.

Candidate agents may be obtained from a wide variety of sources including libraries of synthetic or natural compounds. For example, numerous means are available for random and directed synthesis of a wide variety of organic compounds and biomolecules, including expression of randomized oligonucleotides. Alternatively, libraries of natural compounds in the form of e.g. bacterial, fungal and animal extracts are available or readily produced.

Chemical Libraries: Developments in combinatorial chemistry allow the rapid and economical synthesis of hundreds to thousands of discrete compounds. These compounds are typically arrayed in moderate-sized libraries of small molecules designed for efficient screening. Combinatorial methods can be used to generate unbiased libraries suitable for the identification of novel compounds. In addition, smaller, less diverse libraries can be generated that are descended from a single parent compound with a previously determined biological activity. In either case, the lack of efficient screening systems to specifically target therapeutically relevant biological molecules produced by combinational chemistry such as inhibitors of important enzymes hampers the optimal use of these resources.

A combinatorial chemical library is a collection of diverse chemical compounds generated by either chemical synthesis or biological synthesis, by combining a number of chemical “building blocks,” such as reagents. For example, a linear combinatorial chemical library, such as a polypeptide library, is formed by combining a set of chemical building blocks (amino acids) in a large number of combinations, and potentially in every possible way, for a given compound length (i.e., the number of amino acids in a polypeptide compound). Millions of chemical compounds can be synthesized through such combinatorial mixing of chemical building blocks.

A “library” may comprise from 2 to 50,000,000 diverse member compounds. Preferably, a library comprises at least 48 diverse compounds, preferably 96 or more diverse compounds, more preferably 384 or more diverse compounds, more preferably, 10,000 or more diverse compounds, preferably more than 100,000 diverse members and most preferably more than 1,000,000 diverse member compounds. By “diverse” it is meant that greater than 50% of the compounds in a library have chemical structures that are not identical to any other member of the library. Preferably, greater than 75% of the compounds in a library have chemical structures that are not identical to any other member of the collection, more preferably greater than 90% and most preferably greater than about 99%.

The preparation of combinatorial chemical libraries is well known to those of skill in the art. For reviews, see Thompson et al., Synthesis and application of small molecule libraries, Chem Rev 96:555-600, 1996; Kenan et al., Exploring molecular diversity with combinatorial shape libraries, Trends Biochem Sci 19:57-64, 1994; Janda, Tagged versus untagged libraries: methods for the generation and screening of combinatorial chemical libraries, Proc Natl Acad Sci USA. 91:10779-85, 1994; Lebl et al., One-bead-one-structure combinatorial libraries, Biopolymers 37:177-98, 1995; Eichler et al., Peptide, peptidomimetic, and organic synthetic combinatorial libraries, Med Res Rev. 15:481-96, 1995; Chabala, Solid-phase combinatorial chemistry and novel tagging methods for identifying leads, Curr Opin Biotechnol. 6:632-9, 1995; Dolle, Discovery of enzyme inhibitors through combinatorial chemistry, Mol. Divers. 2:223-36, 1997; Fauchere et al., Peptide and nonpeptide lead discovery using robotically synthesized soluble libraries, Can J. Physiol Pharmacol. 75:683-9, 1997; Eichler et al., Generation and utilization of synthetic combinatorial libraries, Mol Med Today 1: 174-80, 1995; and Kay et al., Identification of enzyme inhibitors from phage-displayed combinatorial peptide libraries, Comb Chem High Throughput Screen 4:535-43, 2001.

Other chemistries for generating chemical diversity libraries can also be used. Such chemistries include, but are not limited to, peptoids (PCT Publication No. WO 91/19735); encoded peptides (PCT Publication WO 93/20242); random bio-oligomers (PCT Publication No. WO 92/00091); benzodiazepines (U.S. Pat. No. 5,288,514); diversomers, such as hydantoins, benzodiazepines and dipeptides (Hobbs, et al., Proc. Nat. Acad. Sci. USA, 90:6909-6913 (1993)); vinylogous polypeptides (Hagihara, et al., J. Amer. Chem. Soc. 114:6568 (1992)); nonpeptidal peptidomimetics with β-D-glucose scaffolding (Hirschmann, et al., J. Amer. Chem. Soc., 114:9217-9218 (1992)); analogous organic syntheses of small compound libraries (Chen, et al., J. Amer. Chem. Soc., 116:2661 (1994)); oligocarbamates (Cho, et al., Science, 261:1303 (1993)); and/or peptidyl phosphonates (Campbell, et al., J. Org. Chem. 59:658 (1994)); nucleic acid libraries (see, Ausubel, Berger and Sambrook, all supra); peptide nucleic acid libraries (see, e.g., U.S. Pat. No. 5,539,083); antibody libraries (see, e.g., Vaughn, et al., Nature Biotechnology, 14(3):309-314 (1996) and PCT/US96/10287); carbohydrate libraries (see, e.g., Liang, et al., Science, 274:1520-1522 (1996) and U.S. Pat. No. 5,593,853); small organic molecule libraries (see, e.g., benzodiazepines, Baum C&E News, January 18, page 33 (1993); isoprenoids (U.S. Pat. No. 5,569,588); thiazolidinones and metathiazanones (U.S. Pat. No. 5,549,974); pyrrolidines (U.S. Pat. Nos. 5,525,735 and 5,519,134); morpholino compounds (U.S. Pat. No. 5,506,337); benzodiazepines (U.S. Pat. No. 5,288,514); and the like.

Devices for the preparation of combinatorial libraries are commercially available (see, e.g., 357 MPS, 390 MPS, Advanced Chem. Tech, Louisville Ky., Symphony, Rainin, Woburn, Mass., 433A Applied Biosystems, Foster City, Calif., 9050 Plus, Millipore, Bedford, Mass.). In addition, numerous combinatorial libraries are themselves commercially available (see, e.g., ComGenex, Princeton, N.J., Asinex, Moscow, Ru, Tripos, Inc., St. Louis, Mo., ChemStar, Ltd., Moscow, RU, 3D Pharmaceuticals, Exton, Pa., Martek Bio sciences, Columbia, Md., etc.).

Small Molecules: Small molecule test compounds can initially be members of an organic or inorganic chemical library. As used herein, “small molecules” refers to small organic or inorganic molecules of molecular weight below about 3,000 Daltons. The small molecules can be natural products or members of a combinatorial chemistry library. A set of diverse molecules should be used to cover a variety of functions such as charge, aromaticity, hydrogen bonding, flexibility, size, length of side chain, hydrophobicity, and rigidity. Combinatorial techniques suitable for synthesizing small molecules are known in the art, e.g., as exemplified by Obrecht and Villalgordo, Solid-Supported Combinatorial and Parallel Synthesis of Small-Molecular-Weight Compound Libraries, Pergamon-Elsevier Science Limited (1998), and include those such as the “split and pool” or “parallel” synthesis techniques, solid-phase and solution-phase techniques, and encoding techniques (see, for example, Czarnik, Curr. Opin. Chem. Bio., 1:60 (1997). In addition, a number of small molecule libraries are commercially available.

The whole procedure can be fully automated. For example, sampling of sample materials may be accomplished with a plurality of steps, which include withdrawing a sample from a sample container and delivering at least a portion of the withdrawn sample to test platform. Sampling may also include additional steps, particularly and preferably, sample preparation steps. In one approach, only one sample is withdrawn into the auto-sampler probe at a time and only one sample resides in the probe at one time. In other embodiments, multiple samples may be drawn into the auto-sampler probe separated by solvents. In still other embodiments, multiple probes may be used in parallel for auto sampling.

In the general case, sampling can be effected manually, in a semi-automatic manner or in an automatic manner. A sample can be withdrawn from a sample container manually, for example, with a pipette or with a syringe-type manual probe, and then manually delivered to a loading port or an injection port of a characterization system. In a semi-automatic protocol, some aspect of the protocol is effected automatically (e.g., delivery), but some other aspect requires manual intervention (e.g., withdrawal of samples from a process control line). Preferably, however, the sample(s) are withdrawn from a sample container and delivered to the characterization system, in a fully automated manner—for example, with an auto-sampler.

In one embodiment, auto-sampling may be done using a microprocessor controlling an automated system (e.g., a robot arm). Preferably, the microprocessor is user-programmable to accommodate libraries of samples having varying arrangements of samples (e.g., square arrays with “n-rows” by “n-columns,” rectangular arrays with “n-rows” by “m-columns,” round arrays, triangular arrays with “r-” by “r-” by “r-” equilateral sides, triangular arrays with “r-base” by “s-” by “s-” isosceles sides, etc., where n, m, r, and s are integers).

Automated sampling of sample materials optionally may be effected with an auto-sampler having a heated injection probe (tip). An example of one such auto sampler is disclosed in U.S. Pat. No. 6,175,409 B1 (incorporated by reference).

According to the present invention, one or more systems, methods or both are used to identify a plurality of sample materials. Though manual or semi-automated systems and methods are possible, preferably an automated system or method is employed. A variety of robotic or automatic systems are available for automatically or programmably providing predetermined motions for handling, contacting, dispensing, or otherwise manipulating materials in solid, fluid liquid or gas form according to a predetermined protocol. Such systems may be adapted or augmented to include a variety of hardware, software or both to assist the systems in determining mechanical properties of materials. Hardware and software for augmenting the robotic systems may include, but are not limited to, sensors, transducers, data acquisition and manipulation hardware, data acquisition and manipulation software and the like. Exemplary robotic systems are commercially available from CAVRO Scientific Instruments (e.g., Model NO. RSP9652) or BioDot (Microdrop Model 3000).

Generally, the automated system includes a suitable protocol design and execution software that can be programmed with information such as synthesis, composition, location information or other information related to a library of materials positioned with respect to a substrate. The protocol design and execution software is typically in communication with robot control software for controlling a robot or other automated apparatus or system. The protocol design and execution software is also in communication with data acquisition hardware/software for collecting data from response measuring hardware. Once the data is collected in the database, analytical software may be used to analyze the data, and more specifically, to determine properties of the candidate drugs, or the data may be analyzed manually.

Data and Analysis: The practice of the present invention may also employ conventional biology methods, software and systems. Computer software products of the invention typically include computer readable medium having computer-executable instructions for performing the logic steps of the method of the invention. Suitable computer readable medium include floppy disk, CD-ROM/DVD/DVD-ROM, hard-disk drive, flash memory, ROM/RAM, magnetic tapes and etc. The computer executable instructions may be written in a suitable computer language or combination of several languages. Basic computational biology methods are described in, for example Setubal and Meidanis et al., Introduction to Computational Biology Methods (PWS Publishing Company, Boston, 1997); Salzberg, Searles, Kasif, (Ed.), Computational Methods in Molecular Biology, (Elsevier, Amsterdam, 1998); Rashidi and Buehler, Bioinformatics Basics: Application in Biological Science and Medicine (CRC Press, London, 2000) and Ouelette and Bzevanis Bioinformatics: A Practical Guide for Analysis of Gene and Proteins (Wiley & Sons, Inc., 2^{nd} ed., 2001). See U.S. Pat. No. 6,420,108.

The present invention may also make use of various computer program products and software for a variety of purposes, such as probe design, management of data, analysis, and instrument operation. See, U.S. Pat. Nos. 5,593,839, 5,795,716, 5,733,729, 5,974,164, 6,066,454, 6,090,555, 6,185,561, 6,188,783, 6,223,127, 6,229,911 and 6,308,170.

Additionally, the present invention relates to embodiments that include methods for providing genetic information over networks such as the Internet.

### Administration of Compositions to Patients

The compositions or agents identified by the methods described herein may be administered to animals including human beings in any suitable formulation. For example, the compositions for modulating protein degradation may be formulated in pharmaceutically acceptable carriers or diluents such as physiological saline or a buffered salt solution. Suitable carriers and diluents can be selected on the basis of mode and route of administration and standard pharmaceutical practice. A description of exemplary pharmaceutically acceptable carriers and diluents, as well as pharmaceutical formulations, can be found in Remington's Pharmaceutical Sciences, a standard text in this field, and in USP/NF. Other substances may be added to the compositions to stabilize and/or preserve the compositions.

The compositions of the invention may be administered to animals by any conventional technique. The compositions may be administered directly to a target site by, for example, surgical delivery to an internal or external target site, or by catheter to a site accessible by a blood vessel. Other methods of delivery, e.g., liposomal delivery or diffusion from a device impregnated with the composition, are known in the art. The compositions may be administered in a single bolus, multiple injections, or by continuous infusion (e.g., intravenously). For parenteral administration, the compositions are preferably formulated in a sterilized pyrogen-free form.

The compounds can be administered with one or more therapies. The chemotherapeutic agents may be administered under a metronomic regimen. As used herein, “metronomic” therapy refers to the administration of continuous low-doses of a therapeutic agent.

Dosage, toxicity and therapeutic efficacy of such compounds can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds that exhibit high therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED₅₀ with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC₅₀ (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

As defined herein, a therapeutically effective amount of a compound (I.e., an effective dosage) means an amount sufficient to produce a therapeutically (e.g., clinically) desirable result. The compositions can be administered one from one or more times per day to one or more times per week; including once every other day. The skilled artisan will appreciate that certain factors can influence the dosage and timing required to effectively treat a subject, including but not limited to the severity of the disease or disorder, previous treatments, the general health and/or age of the subject, and other diseases present. Moreover, treatment of a subject with a therapeutically effective amount of the compounds of the invention can include a single treatment or a series of treatments.

### Formulations

While it is possible for a composition to be administered alone, it is preferable to present it as a pharmaceutical formulation. The active ingredient may comprise, for topical administration, from 0.001% to 10% w/w, e.g., from 1% to 2% by weight of the formulation, although it may comprise as much as 10% w/w but preferably not in excess of 5% w/w and more preferably from 0.1% to 1% w/w of the formulation. The topical formulations of the present invention, comprise an active ingredient together with one or more acceptable carrier(s) therefor and optionally any other therapeutic ingredients(s). The carrier(s) must be “acceptable” in the sense of being compatible with the other ingredients of the formulation and not deleterious to the recipient thereof.

Formulations suitable for topical administration include liquid or semi-liquid preparations suitable for penetration through the skin to the site of where treatment is required, such as liniments, lotions, creams, ointments or pastes, and drops suitable for administration to the eye, ear, or nose. Drops according to the present invention may comprise sterile aqueous or oily solutions or suspensions and may be prepared by dissolving the active ingredient in a suitable aqueous solution of a bactericidal and/or fungicidal agent and/or any other suitable preservative, and preferably including a surface active agent. The resulting solution may then be clarified and sterilized by filtration and transferred to the container by an aseptic technique. Examples of bactericidal and fungicidal agents suitable for inclusion in the drops are phenylmercuric nitrate or acetate (0.002%), benzalkonium chloride (0.01%) and chlorhexidine acetate (0.01%). Suitable solvents for the preparation of an oily solution include glycerol, diluted alcohol and propylene glycol.

Lotions according to the present invention include those suitable for application to the skin or eye. An eye lotion may comprise a sterile aqueous solution optionally containing a bactericide and may be prepared by methods similar to those for the preparation of drops. Lotions or liniments for application to the skin may also include an agent to hasten drying and to cool the skin, such as an alcohol or acetone, and/or a moisturizer such as glycerol or an oil such as castor oil or arachis oil.

Creams, ointments or pastes according to the present invention are semi-solid formulations of the active ingredient for external application. They may be made by mixing the active ingredient in finely-divided or powdered form, alone or in solution or suspension in an aqueous or non-aqueous fluid, with the aid of suitable machinery, with a greasy or non-greasy basis. The basis may comprise hydrocarbons such as hard, soft or liquid paraffin, glycerol, beeswax, a metallic soap; a mucilage; an oil of natural origin such as almond, corn, arachis, castor or olive oil; wool fat or its derivatives, or a fatty acid such as stearic or oleic acid together with an alcohol such as propylene glycol or macrogels. The formulation may incorporate any suitable surface active agent such as an anionic, cationic or non-ionic surface active such as sorbitan esters or polyoxyethylene derivatives thereof. Suspending agents such as natural gums, cellulose derivatives or inorganic materials such as silicaceous silicas, and other ingredients such as lanolin, may also be included.

While various embodiments of the present invention have been described above, it should be understood that they have been presented by way of example only, and not limitation. Numerous changes to the disclosed embodiments can be made in accordance with the disclosure herein without departing from the spirit or scope of the invention. Thus, the breadth and scope of the present invention should not be limited by any of the above described embodiments.

All documents mentioned herein are incorporated herein by reference. All publications and patent documents cited in this application are incorporated by reference for all purposes to the same extent as if each individual publication or patent document were so individually denoted. By their citation of various references in this document, Applicants do not admit any particular reference is “prior art” to their invention. Embodiments of inventive compositions and methods are illustrated in the following examples.

### EXAMPLES

The following non-limiting Examples serve to illustrate selected embodiments of the invention. It will be appreciated that variations in proportions and alternatives in elements of the components shown will be apparent to those skilled in the art and are within the scope of embodiments of the present invention.

### Example 1

### CD2AP Proteolysis and Progression of Kidney Disease

Methods

Cell culture and transient transfection. Mouse podocytes were cultured as described previously (Mundel, P. et al. Exp. Cell Res. 236, 248-258 (1997)). HEK293 cells were maintained and transfected as previously reported (Reiser, J. et al. Nat. Genet. 37, 739-744 (2005)).

Antibodies. The following primary antibodies were used: mouse anti-actin (Sigma), mouse anti-dynamin (Hudy 1; Upstate Biotechnology), mouse anti-GAPDH (Abcam), rat anti-LAMP2 (Developmental Studies Hybridoma Bank), FITC-conjugated phalloidin (Sigma), rabbit anti-WT1 (Santa Cruz Biotechnology) rabbit anti-alpha-actinin-431, rabbit anti-cathepsin L32, rabbit anti-CD2AP28, rabbit anti-dendrin and mouse anti-synaptopodin.

Computing the scores of endopeptidase cleavage sites. To assess the susceptibility of CD2AP for cleavage by cathepsin L in silico, the ‘Prediction of Endopeptidase Substrates’ (PEPS) bioinformatics tool was utilized (Lohmüller, T. et al. Biol. Chem. 384, 899-909 (2003)). A score above the threshold of 0.01 estimates protein sequences to be within 100 peptide motifs (out of 10000).

Immunohistochemistry and immunoblotting. Immunocytochemical analysis of cultured podocytes was performed as described previously (Mundel, P. et al. Exp. Cell Res. 236, 248-258 (1997)). SDS-PAGE and Western blotting were done with the modification that used Invitrogen's blot module (XCell Sure-Lock Tank), gels (4-12% NuPAGE Bis-Tris), running (MES or MOPS) and transfer buffers.

Communoprecipitation studies. Recombinant mouse FLAG-dendrin were expressed with GFP-tagged CD2AP variants (full-length CD2AP, CD2AP-NH, CD2AP-COOH) in HEK293 cells. FLAG fusion proteins were immunoprecipitated from cell lysates using anti-FLAG-M2 beads (Sigma) and analyzed eluates by immunoblotting using antibodies to FLAG (Sigma) or GFP (Invitrogen).

Deletion of CD2AP cleavage site LSAAE. Deletion of the cathepsin L cleavage site LSAAE from the CD2AP amino acid sequence was done using the QuickChange II Site Directed Mutagenesis kit (Stratagene) according to the manufacturer's instructions.

Isolation and processing of glomeruli. Glomeruli were isolated from kidneys of 8-12 weeks old LPS- and PBS-treated (control) mice using a sequential sieve technique with mesh sizes of 180, 100, and 71 μm. The fraction collected from the 71-μm sieve was maintained for soup/pellet fractionation. Isolated glomeruli were homogenized in buffer containing 20 mM HEPES pH 7.5, 100 mM NaCl, 1 mM MgCl₂, 1 mM PMSF, protease inhibitors (Roche), calpain inhibitor (Calbiochem), and E-64d (Calbiochem) using Dounce homogenizer. Subsequently, cytosol was centrifuged for 10 min at 4,600 g. Proteins were solubilized by 1% Triton X-100, 1 hour at 4° C., before it was spun at 70,000 g for 1 hour.

Cathepsin L activity assay. Subcellular sites of cathepsin L and cathepsin B activity in glomerular extracts were visualized by a fluorogenic substrate, CV—(FR)₂, which emits light upon cleavage by cathepsin L or cathepsin B (Biomol). Cathepsin L inhibitor Z-FF-FMK (Calbiochem) which does not inhibit cathepsin B was used for specific inhibition of cathepsin L.

In vivo gene delivery. Cathepsin L plasmids encoding short and long cathepsin L, were introduced into mice (n>10, each construct) using the TransIT in vivo gene delivery system (Mirus). For serum nephritis experiments, FLAG-tagged encoding wild type and cathepsin L cleavage resistant CD2AP plasmids were delivered twice by tail vein injection on day 8 and 10 after serum nephritis induction. Expression of plasmids were monitored in kidney cortex slices by immunoblot.

Purification of CD2AP protein. FLAG-CD2AP was expressed in HEK293T cells, immobilized on anti-FLAG M2 agarose (Sigma) and eluted with FLAG-peptide (Sigma).

Proteolytic processing of CD2AP by cathepsin L. CD2AP was diluted in buffer containing 200 mM NaCl, 10 mM HEPES pH 7.0, 2 mM EGTA, 1 mM MgCl₂, and 1 mM DTT. When indicated, 20 μM cathepsin L inhibitor Z-FF-FMK was added. The reaction was initiated by addition of 0.5 μl of purified cathepsin L (specific activity 4.13 U/mg of protein from Sigma), and samples were placed at 37° C. in the water bath for 10 to 30 min. Total assay volume was 20 μl. The reaction was terminated with addition of E-64d inhibitor (Sigma) and sample buffer. For Western blot analysis, 5 ml of the samples was run on 10% SDS-PAGE.

Chemical Crosslinking and Native PAGE. Chemical crosslinking was performed according to standard protocols with the DTSSP crosslinking reagent (Pierce). Native PAGE was performed with the NativePAGE system (Invitrogen) according to the manufacturer's instructions.

Electron Microscopy and Image Reconstruction. Aliquots (˜5 μl of 50 μg/ml protein) were allowed to adhere for 2-5 min to carbon-coated copper grids and then stained with 2% uranyl acetate. Images were recorded under minimum electron dose conditions using a CM10 electron microscope (Philips Electron Optics). Images were recorded on Kodak 4489 film at a nominal magnification of 52,000 using 100 kV electrons. Micrographs were digitized with a Coolscan 9000 scanner (Nikon) at 8 bits per pixel and 6.35 μm per pixel, subsequently averaged to 12.7 μm per pixel. The optical density for each negative was adjusted to give a mean value of ˜127 over the total range of 0 to 255. Image processing was performed with the EMAN suite. A total of 5996 particles were selected from 25 micrographs. The CTF for each micrograph was manually determined with the EMAN program ctfit and phase corrections applied to the selected particles. Initial models were generated using the EMAN routine startcsym, which conducts a symmetry search of the particles for four-fold and mirror symmetry, representing top and side views, respectively. These orthogonal projections are subsequently aligned with a common-lines algorithm and back-projected to generate a 3D structure. Models were subjected to refinement with C4 symmetry imposed with an angular increment of 6°. The isosurface for the final model was determined from the molecular weight of the tetramer (300 kD) which encloses a volume of 370,000 Å3 using a protein partial specific volume of 0.74 cm³/g. Atomic coordinates for the SH3 domain and tetrameric coiled-coil domain were visually fitted within the EM map.

Animal model of experimental glomerulonephritis. Serum nephritis was induced as described previously (Monkawa, T. et al. Nephron Exp. Nephrol. 102, e8-e18 (2006)). Injection of anti-GBM serum on day 0 and 1 induced glomerulonephritis in wild type and cathepsin L KO mice (both C57B16), aged 8-14 weeks with a sheep antibody reactive to rabbit glomeruli (12.5 mg/20 g body wt intraperitoneal injection per day for two consecutive days). Mice were sacrificed at day 14 (n=5 in each group).

Mouse phenotyping. Freshly harvested kidneys were fixed in 4% PFA (Electron Microscopy Sciences) solution. They were then embedded in paraffin and 2 micron sections cut and stained with hematoxylin and eosin (H&E), periodic acid-Schiff (PAS) reagent or methenamine-silver stain. The sections were examined in a blinded manner and scored for glomerular and other renal changes. Glomerular lesion scores were assigned on a 4 point scale based on the number of glomeruli involved and the severity of the lesions (1, 1 score; 2, 2-3 scores; 3, 3 and above scores; 4, 3 and above with confluency). Overall lesion scores included focal hypercellularity, glomerulosclerosis (FSGS), crescent formation, and podocyte apoptosis. Thirty glomeruli in each kidney were examined. Urine microalbumin was assessed by the densitometric analysis of the Bis-Tris gels loaded with the standard BSA (Bio-Rad Laboratories) and the urine samples. The urinary creatinine measurement was carried out using a colorimetric end-point assay with a commercial kit (Cayman Chemical).

Human kidney biopsy staining. Human glomerular biopsies (Control, Minimal Change Disease, and Focal Segmental Glomerulosclerosis) were stained with N- and C-terminal CD2AP antibodies following standard protocols.

Statistical analysis. Statistical analysis was performed by Student's t-test with the level of significance set at P<0.05. Data are reported as mean values+/− standard error of the means.

Results:

In this study, the identification of the cytoskeletal adaptor protein CD2AP as a cleavage target for cytoplasmic cathepsin L is described. CD2AP is a scaffolding protein required for homeostasis of podocytes. Homozygous CD2AP mutation or haploinsufficiency of the human CD2AP gene predispose to renal disease and mice lacking CD2AP develop progressive kidney failure. Similarly, mice with bigenic haploinsufficiency of synaptopodin and CD2AP develop disease consistent with progressive renal failure. CD2AP carries a special weight in the maintenance of podocyte structure and function. This example, identified CD2AP as a cleavage target of cathepsin L and the structure of CD2AP at 21 Å resolution was characterize as a cuboid tetrameric multi-adapter that exposed two accessible cathepsin L cleavage sites. The limited remodeling of CD2AP by cytoplasmic cathepsin L leaves behind a C-terminal core fragment that is structurally competent but can no longer bind dendrin, a protein which promotes podocyte apoptosis in the presence of transforming growth factor-β (TGF-β) once it enters the nucleus. Cathepsin L controls the proteolysis of dynamin and synaptopodin, events that are contributing to the development of podocyte FP effacement and proteinuria. The identification of the structure of CD2AP and its role as a cathepsin L substrate unraveled important aspects of kidney disease progression. It provides insights into the mechanisms of kidney disease pathogenesis and progression.

CD2AP is proteolyzed by cathepsin L: The computer algorithm PEPS served to identify that cathepsin L cleavage targets dynamin and synaptopodin. A reduction of CD2AP staining was noted at cell-cell junctions in cultured podocytes that express high levels of cathepsin L but not in podocytes that lack cathepsin L. The PEPS-algorithm was applied to potentially identify cathepsin L cleavage sites within the CD2AP amino acid sequence. Eleven putative cathepsin L sites within the CD2AP mouse and human protein sequence (Table 1) were identified.

TABLE 1^{A}

| Cleavage | Starting | Prediction |
|---|---|---|
| sequence | amino acid | score |
| EIIRV | 23 | 0.02462 |
| LIVGV | 128 | 0.02502 |
| APGSV | 199 | 0.02700 |
| KIRGI | 208 | 0.02242 |
| QPLGS | 247 | 0.03190 |
| ELGKE | 311 | 0.02756 |
| LSAAE | 352 | 0.02280 |
| AFVAR | 462 | 0.02214 |
| LPGRF | 499 | 0.02796 |
| ELAKA | 565 | 0.02200 |
| ELRKE | 606 | 0.02589 |

^{A}Cathepsin L cleavage sites on CD2AP amino acid sequence which was identified by the computer-based prediction of endopeptidase cleavage sites (PEPS) algorithm. PEPS yielded a total of 11 putative cleavage sites with CD2AP. The PEPS prediction score is the sum of the amino acid scores in a block of 5 consecutive amino acids of the test protein in the cleavage matrix (P4-P1′ or P3-P2′). The PEPS algorithm screens over the protein sequence and gives the sum score for any peptide of 5 amino acids within a protein sequence. These PEPS sum scores are further compared the scores to all other 5 amino acid peptides in the proteome: A cathepsin L PEPS score of 0.02 denotes a 80% likelihood to be cleaved (based on the mouse proteome); a score of 0.04 denotes a chance for cleavage of 99% (the fit is not linear). Hence the multiple potential cathepsin L cleavage sites in CD2AP (PEPS scores ranging from 0.022 to 0.0319) implicate a likelihood for cleavage of approximately 90%.

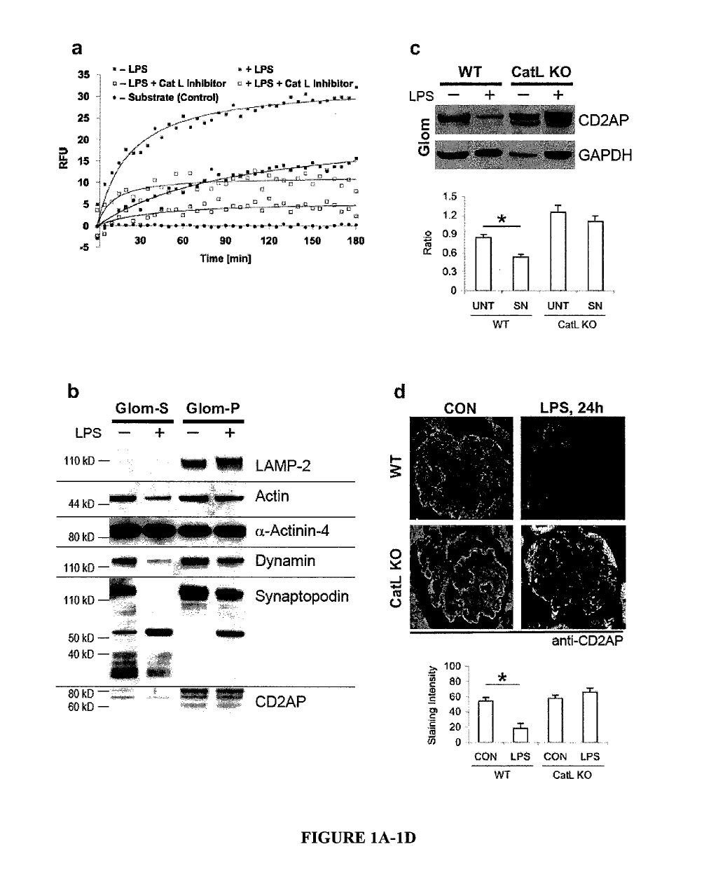

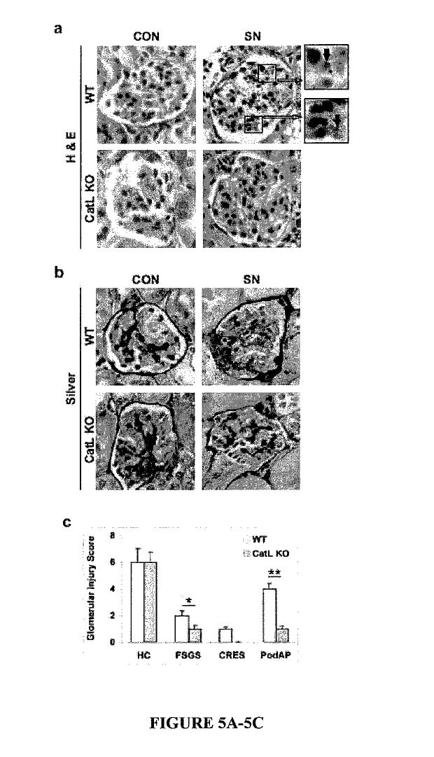

To test the significance of this prediction, glomeruli were isolated from control mice and animals that were injected with low-dose lipopolysaccharide (LPS), a treatment causing high levels of cytosolic cathepsin L in podocytes. The tissue samples were further processed to obtain cytosolic and membrane-bound glomerular extracts (FIG. 1A). A fluorescent enzymatic assay showed strong cathepsin L activity in the LPS treated cytosolic extract of glomeruli that could be inhibited by co-incubation with a specific cathepsin L inhibitor. In contrast, the same fractions had a much lower activity of cytosolic cathepsin L without prior treatment of the animals with LPS. These glomerular protein fractions were utilized in immunoblots for the cathepsin L target proteins dynamin and synaptopodin as well as for CD2AP using an anti-serum against CD2AP that recognized the N-terminal SH3 domains of CD2AP (FIG. 1B, FIG. 5E). All target proteins were found to be reduced in the cytosolic fraction but not in the membrane bound fraction. By comparison, α-actinin-4 was not reduced in the cytosolic as well as the pelleted glomerular fraction. To further prove that reduction of CD2AP stems from cathepsin L, cathepsin L knockout mice were utilized in the analysis of glomerular lysates for CD2AP levels. A significant reduction of CD2AP was found after LPS treatment but did not see this effect in glomeruli in which cathepsin L was absent (FIG. 1C). Immunostaining was also carried out in glomeruli during transiently high levels of cathepsin L, e.g. after LPS treatment (FIG. 1D), puromycin treatment and after gene transfer of cytosolic cathepsin L into podocytes. Following LPS injection, CD2AP protein staining was decreased after 24 hours in wild type mice but not in cathepsin L knockout mice evidencing proteolysis of CD2AP (FIG. 1D). CD2AP reduction was also observed in cultured podocytes that were either treated with LPS or PAN, both conditions with high levels of cytosolic cathepsin L. All together, these data strongly evidence that cytosolic cathepsin L proteolyses CD2AP in vivo.

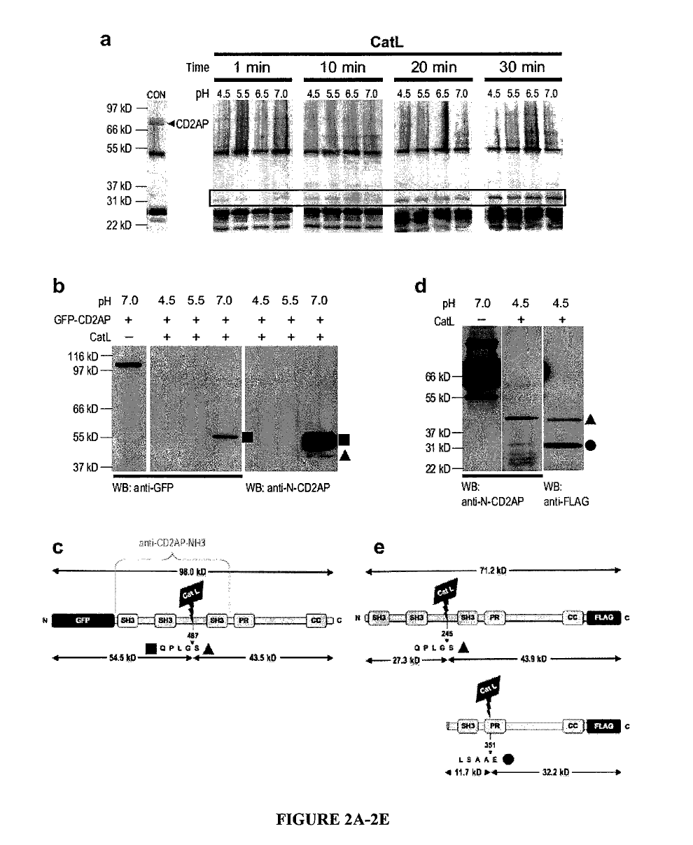

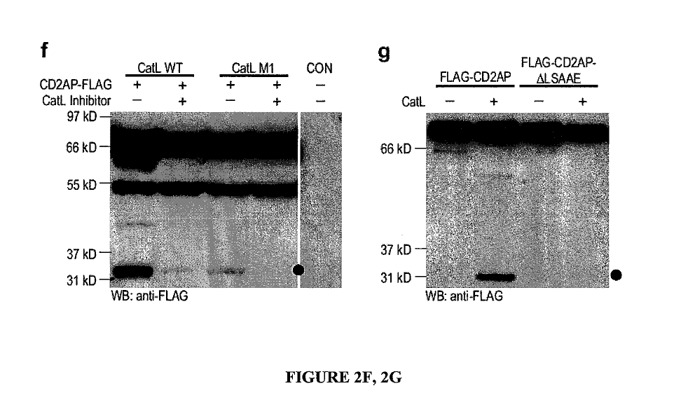

Cathepsin L processes CD2AP into a C-terminal 32 kD fragment (p32): CD2AP protein was purified from transfected mammalian cells (HEK 293) and in vitro cleavage assays using were performed using purified cathepsin L enzyme at various pH ranging from acidic (lysosomal) to neutral pH 7.0 (FIG. 2A). pH 7.0 was determined as the pH that is present in the podocyte cytosol under normal and LPS conditions using Nuclear Magnetic Resonance Spectroscopy analysis. Cathepsin L cleaved CD2AP strongly at acidic pH, a finding that is in line with its potent role in lysosomes where cleavage occurs on random targets and nonspecifically. By contrast, cleavage assays performed at neutral conditions (pH 7.0) yielded a stable 32 kD CD2AP fragment (p32) that was detectable by silver stain following electrophoretic separation of the cleaved CD2AP protein fragments. Of note, p32 increased with incubation time of CD2AP with cathepsin L at pH 7.0. To better characterize the cleavage of CD2AP, GFP- and FLAG-tagged fusion proteins were generated that were exposed to cathepsin L (FIGS. 2B-2E). An N-terminal tagged GFP-CD2AP fusion protein (98 kD) was expressed in HEK 293 cells, purified and subjected to cleavage assays with cathepsin L enzyme (FIG. 2B). Cleavage of CD2AP at pH 4.5 and 5.5 led to the complete digestion of the protein. However, at pH 7.0, a CD2AP cleavage fragment was identified consistent with the predicted major cathepsin L cleavage site QPLGS (Table 1, FIG. 2C). At neutral pH, CD2AP was cleaved into a stable 55 kD fragment as detected with an anti-GFP antibody. The same fragment was detected with the CD2AP antiserum raised against the SH3 domains of CD2AP. The CD2AP antiserum also reacted with a N-terminal 44 kD fragment (explained by the affinity of the antibody to the third SH3 domain). Of note, the anti-GFP antibody as well as the anti-CD2AP antibody could not detect C-terminal p32 (compare with FIG. 5E). Additional cathepsin L cleavage experiments were performed using a C-terminal FLAG-tagged CD2AP (71 kD) expressed in HEK 293 cells and immobilized on FLAG beads before digestion with cathepsin L enzyme (FIG. 2D). This experiment yielded a C-terminal 44 kD fragment of CD2AP, which could be detected by CD2AP antiserum (raised against the three SH3 domains) and by anti-FLAG antibody. The anti-CD2AP antiserum also detected a band at 27 kD. These fragments were again consistent with the CD2AP cleavage site QPLGS (FIG. 2E). The anti-FLAG antibody also detected a strong band corresponding to p32 fragment which could be matched to the secondary CD2AP cleavage site LSAAE (FIG. 2E). In summary, these data provide evidence for the p32 C-terminal fragment of CD2AP to be a stable cleavage product of CD2AP in podocytes at physiological cytosolic pH 7.0.

Next, it was investigated whether cytosolic cathepsin L in cells is sufficient to process CD2AP. CD2AP-FLAG expressing HEK293 cells were co-transfected with WT cathepsin L mRNA which generates cytosolic and lysosomal cathepsin L protein and a cathepsin L construct that contains a deletion of the first AUG site and thus encodes selectively for the cytosolic form of cathepsin L. The experiments were performed in the presence or absence of a specific cathepsin L inhibitor (FIG. 2F). WT cathepsin L led to cleavage of CD2AP yielding p32. The generation of p32 could be prevented by co-incubation of transfected HEK 293 cells with a specific cathepsin L inhibitor. More importantly, transfection of cytosolic cathepsin L alone was sufficient to cleave CD2AP resulting in the production of p32 (FIG. 2F). This cleavage was cathepsin L dependent since it could be blocked by the addition of cathepsin L inhibitor. The stable p32 fragment was identified in the cleavage assays at neutral pH (FIG. 2A). In addition, this fragment was generated in HEK 293 cells using cytosolic cathepsin L. Therefore, this fragment was regarded as an end-product generated through cytosolic cathepsin L cleavage. Next the LSAAE cleavage site in the CD2AP protein was deleted. In absence of this cleavage site, the characteristic p32 fragment was no longer observed after enzymatic digest with cathepsin L enzyme (FIG. 2G) indicating that removal of the LSAAE site in CD2AP protected from cathepsin L mediated enzymatic processing of CD2AP into p32.

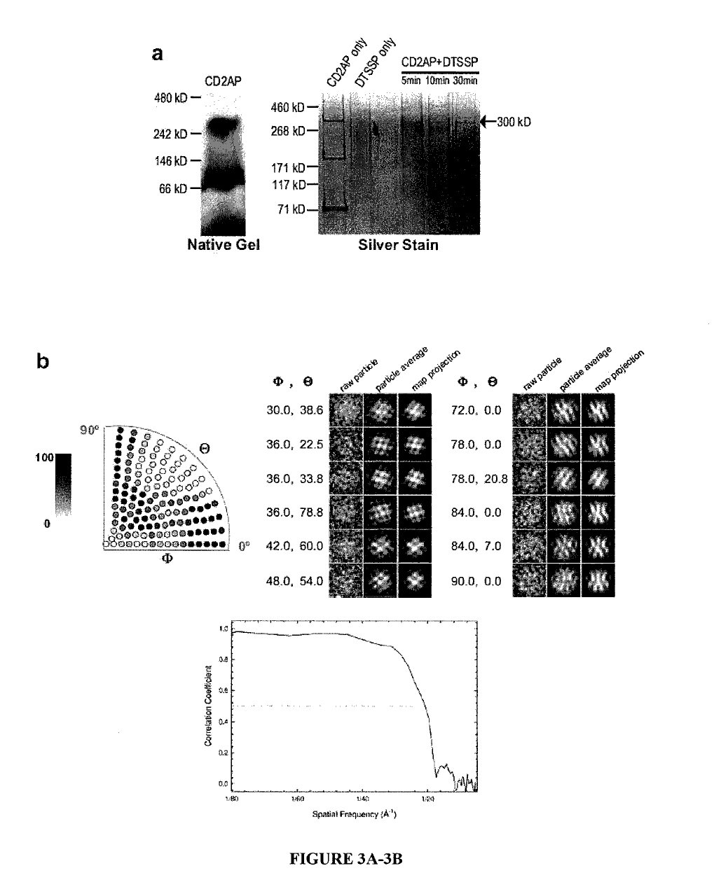

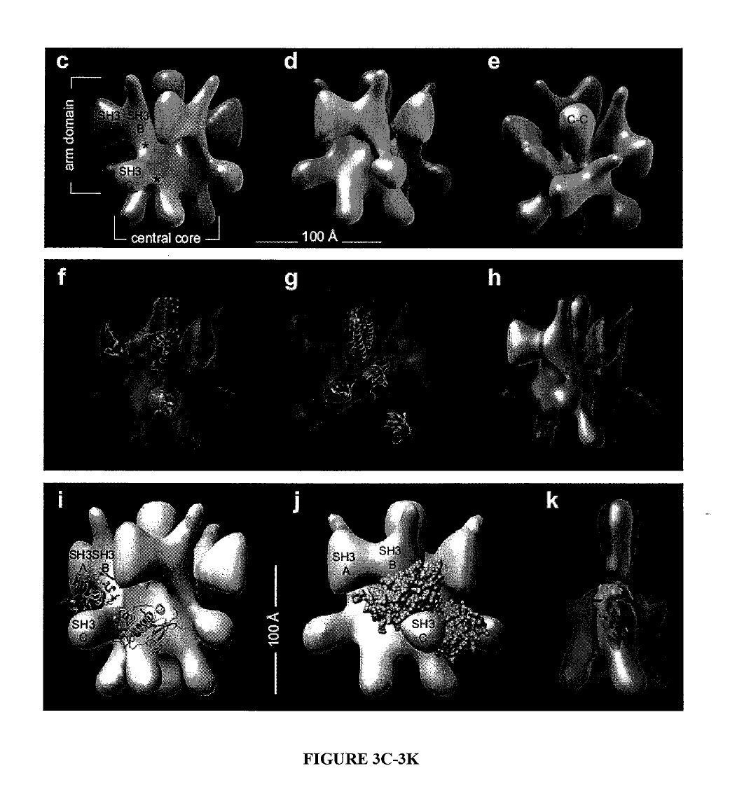

CD2AP is a tetramer that exposes cleavage sites QPLGS and LSAAE: Cytosolic cathepsin L mediated cleaving of dynamin and synaptopodin are protected from cleavage through higher order assembly of dynamin or through serine-threonine phosphorylation dependent binding of 14-3-3 protein to synaptopodin that in turn blocks cleavage sites from the exposure to cathepsin L. Higher molecular complexes of approximately 300 kD were identified when purified CD2AP was separated in native gels or after chemical cross-linking (FIG. 3A). This led to next experiment which would characterize CD2AP protein multimers by electron microscope to gain insights into its structure that may help for a better understanding of the enzymatic susceptibility of CD2AP. Visual inspection of negatively stained micrographs of purified CD2AP revealed dispersed, roughly spherical molecular complexes with overall dimensions compatible with a tetrameric organization. Three dimensional image reconstruction of the particles using the EMAN processing suite was undertaken with four-fold rotational symmetry imposed during the refinement (FIG. 3B). A Fourier shell correlation computed between maps generated from a split data set indicate that the map has a resolution of 21 Å using the 50% correlation criterion (FIG. 3B). The resulting structural map reveals a cubic-like molecule with four of the faces related by the rotational symmetry (FIGS. 3C-3E). The structure is not very compact and stain has penetrated throughout to reveal clearly identifiable domains (FIGS. 3F-3H). The overall organization consists of a central core, broad at one end but tapering to a straight cylinder coincident with the fourfold axis at the other. The central core is surrounded by four symmetry related motifs each containing three globular domains. The individual domains within the structure were assigned by performing comparison of the map density with known homologous structures. In the case of CD2AP, this helped to identify the three N-terminal SH3 domains and the extreme C-terminal coiled-coil domain (FIGS. 3F-3H). Both experimentally confirmed cathepsin L cleavage sites QPLGS and LSAAE were exposed at the connecting area of the SH3 domains with the CD2AP core (FIG. 3C, asterisks). Cathepsin L enzyme fits well into the pockets of the CD2AP-SH3 domains (FIGS. 3I, 3J) to process CD2AP into a structurally competent protein core that lacks the SH3 domains (FIG. 3K).

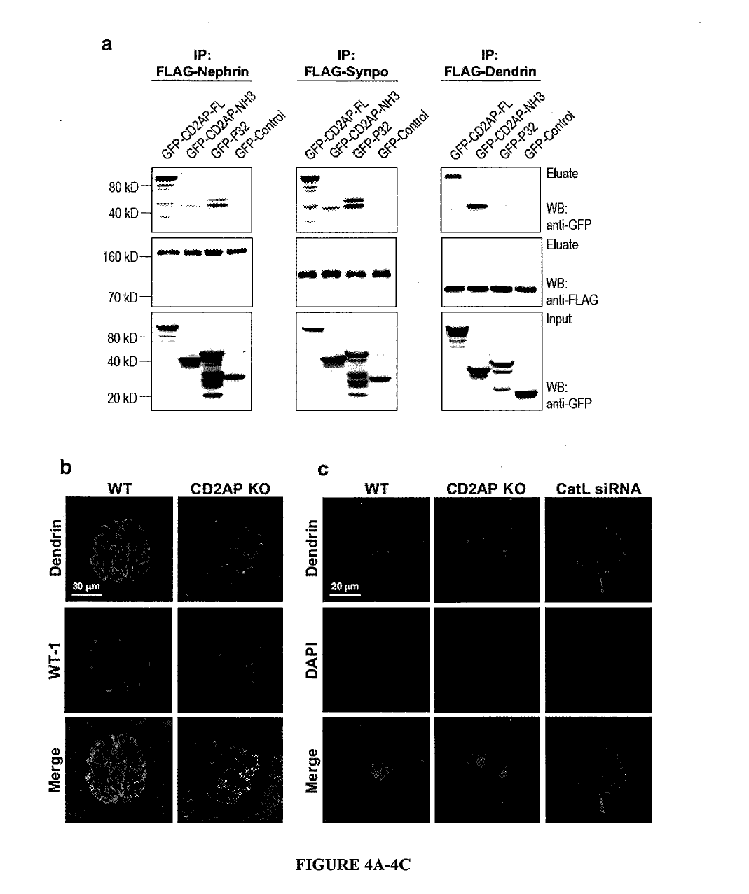

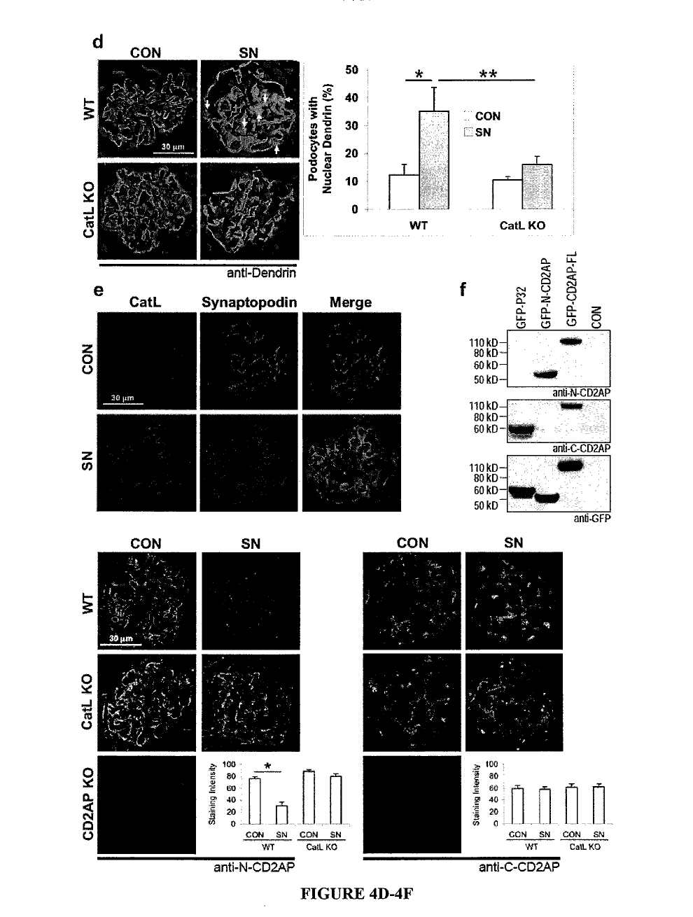

Function of the C-terminal CD2AP fragment (p32): Next the consequences of CD2AP proteolysis into p32 were explored. While major changes were not observed in endocytosis in the presence of p32, the known interactions that CD2AP undergoes with the actin organizing protein synaptopodin, the slit diaphragm protein nephrin and dendrin were analyzed, that under physiological conditions inhibits podocyte apoptosis through interaction with CD2AP at cell-cell junctions. During glomerular injury such as in serum nephritis, dendrin can translocate to the nucleus in podocytes to promote apoptosis. To investigate whether p32 can still bind to synaptopodin, nephrin and dendrin, co-immunoprecipitation studies were performed with GFP and FLAG-tagged protein combinations expressed in HEK293 cells (FIG. 4A). Both N- and p32 fragments of CD2AP were still able to bind synaptopodin and the slit diaphragm molecule nephrin evidencing that the generated CD2AP fragments may at least partially maintain podocyte cytoskeletal function. However, while N-terminal and full length CD2AP still bind dendrin, the p32 fragment of CD2AP was incapable to undergo this interaction.

Dendrin is found in the nucleus of CD2AP null mice and podocytes: Dendrin is a slit diaphragm protein that promotes TGF-β induced podocyte apoptosis through relocating from the cell periphery to the nucleus. Furthermore, CD2AP^{-/-} podocytes are more susceptible to TGF-β mediated apoptosis and CD2AP^{-/-} mice are born with normal podocyte FP. However, these mice display elevated levels of glomerular TGF-β and develop severe progressive glomerular disease starting approximately at 4 weeks of age 12. The disease in these mice is characterized by massive podocyte apoptosis and glomerular sclerosis within 7 weeks. It was hypothesized that dendrin that cannot be bound by CD2AP will be present in the nucleus of podocytes and compared the localization of CD2AP in WT and CD2AP^{-/-} podocytes in vivo. In 5 weeks old WT mice, dendrin was found in podocytes following a classical capillary loop pattern outside nuclei as shown by double labeling with WT-1 (FIG. 4B). In contrast, 5 weeks old CD2AP^{-/-} mice that were developing severe glomerular disease, showed dendrin labeling in podocyte nuclei overlapping with the expression of WT-1 (FIG. 4B). Also studied was the expression of dendrin in WT, CD2AP^{-/-} and Cathepsin L knockdown cultured podocytes (FIG. 4C). While dendrin was absent from WT and cathepsin L knockdown nuclei, yet was mainly located at the plasma membrane and in the cytoplasm, dendrin was found in the nuclei of CD2AP^{-/-} podocytes (FIG. 4C). In sum, the data evidences that the absence of CD2AP or the limited proteolysis of CD2AP into p32 releases dendrin and allows its transfer to the nucleus.

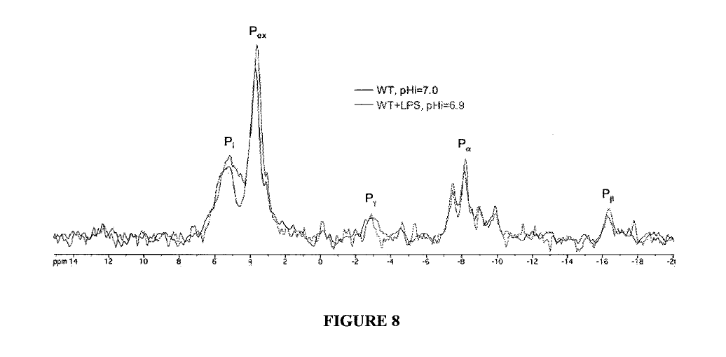

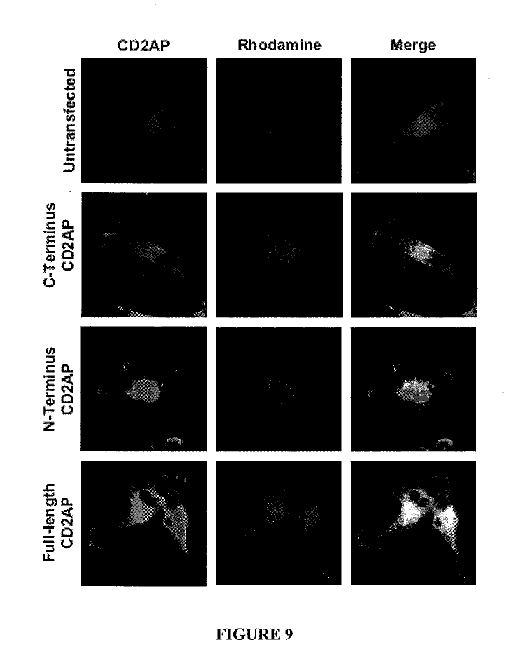

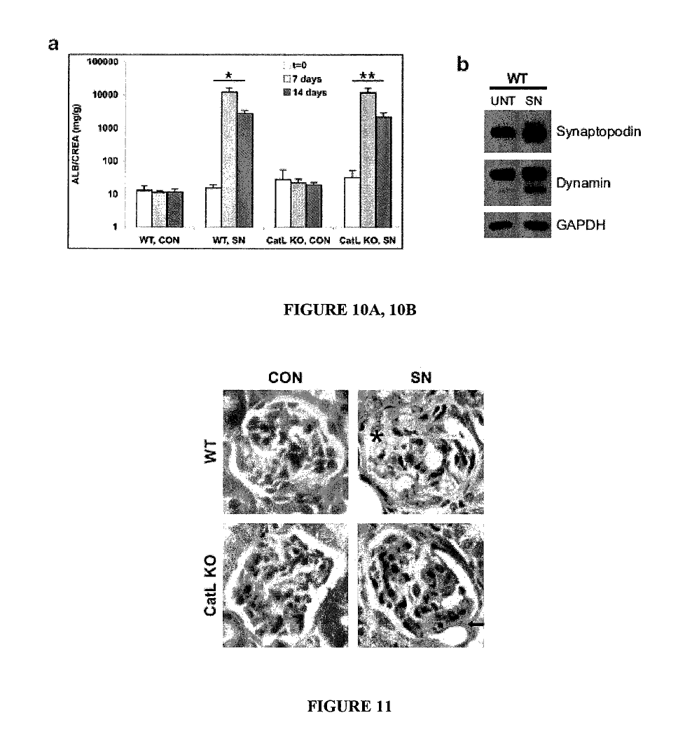

Cathepsin L proteolyses CD2AP in a progressive model of renal disease: If lack of CD2AP allows dendrin to enter the nucleus in progressive renal disease occurring in CD2AP knockout mice (FIG. 9B), it was hypothesized that a similar finding in a progressive kidney disease model where p32 is generated, would be found. Based on this hypothesis, the serum nephritis mouse model was utilized in which injection of an antibody that reacts with the glomerular basement membrane causes features of advancing glomerular disease such as crescents and podocyte apoptosis. Moreover, this model displays nuclear relocation of dendrin in podocytes. After induction of serum nephritis, we found a significant relocation of dendrin into podocyte nuclei of wild type mice (P=0.0008 vs. CatL KO, SN), a response that was largely absent in cathepsin L knockout mice (FIG. 4D).

Cathepsin L was induced in podocytes during serum nephritis in wild type mice as shown by double labeling with synaptopodin but was not detected in cathepsin L knockout mice (FIG. 4E). It was next analyzed if there was a loss of the N-terminal SH3 domains and an unchanged expression of C-terminal CD2AP that included p32 in glomeruli of wild type mice. Using two different antibodies for CD2AP, 1) anti-CD2AP N-terminal (recognizes SH3 domains) and 2) anti-CD2AP C-terminus (recognizes p32), it was found that N-terminal CD2AP was significantly reduced during serum nephritis in wild type mice but not in cathepsin L knockout mice (FIG. 4F). In addition, there was no reduction in C-terminal CD2AP staining consistent with a stable C-terminal CD2AP (FIG. 4F). The specificity for the antibodies was proven by immunoblot from HEK 293 cell lysates that express GFP-tagged CD2AP fragments (FIG. 4F). In summary, this data shows that serum nephritis is associated with cytosolic cathepsin L induction in podocytes that leads to proteolysis of CD2AP N-terminus but stable C-terminal fragment (p32) and the release of dendrin to the podocyte nucleus.

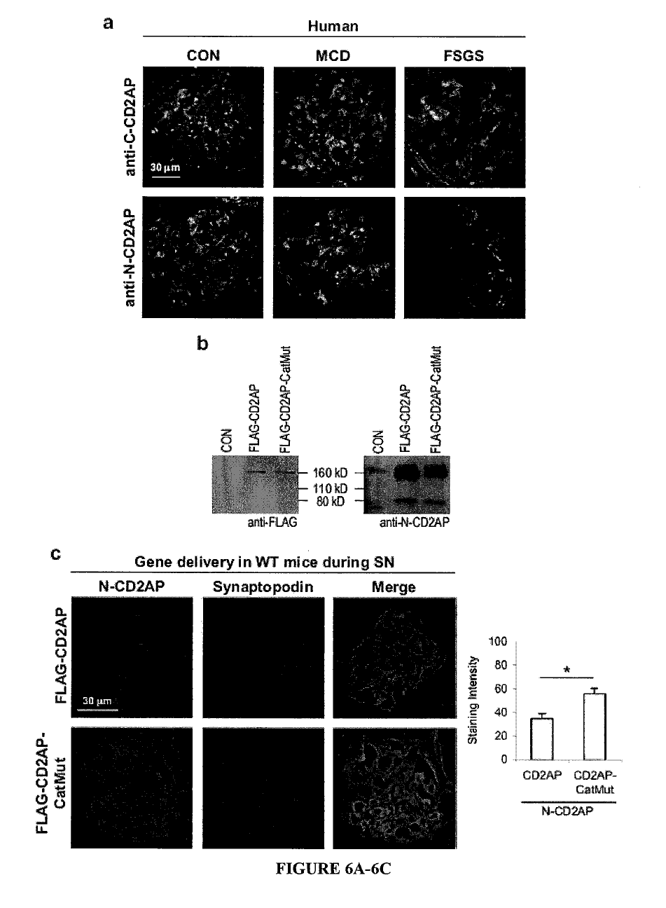

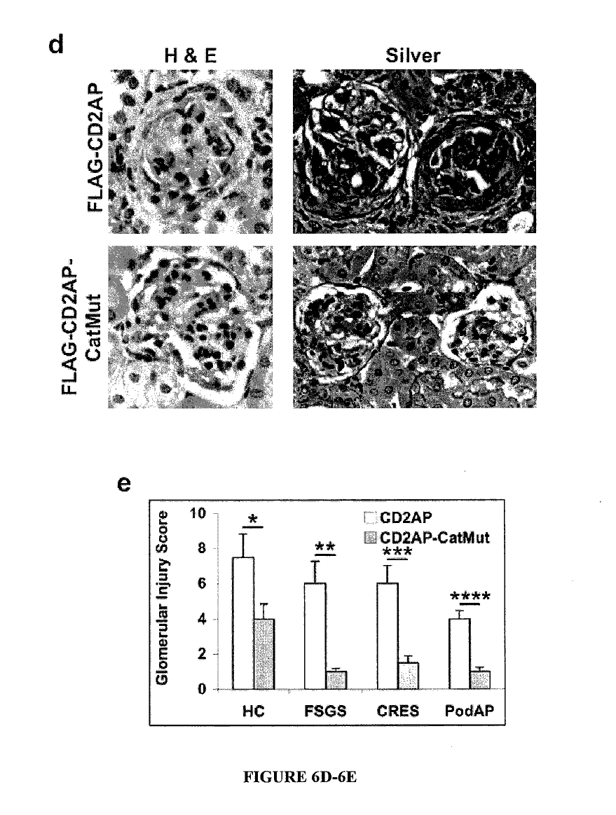

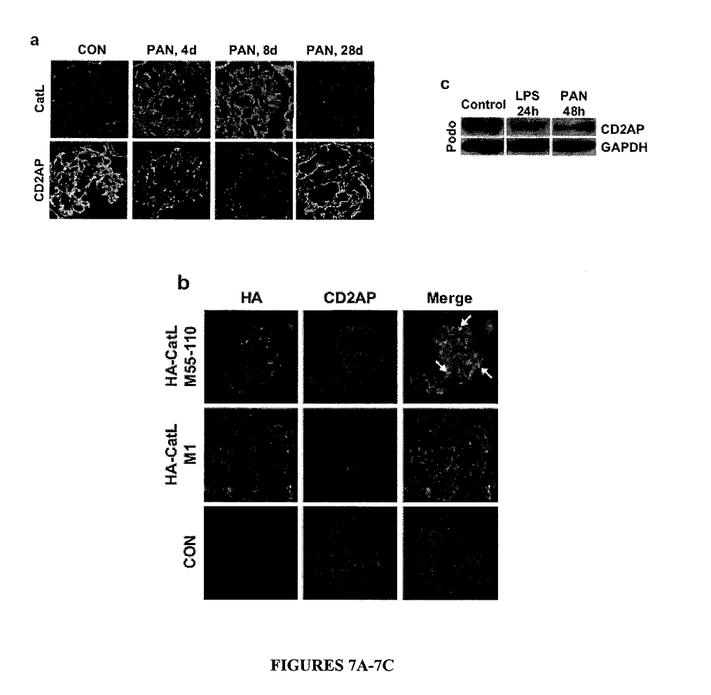

Reduction of renal disease progression in mice lacking cathepsin L: The absence of cathepsin L ensures significantly higher expression of CD2AP during serum nephritis (FIG. 4F). Both, wild type and cathepsin L knockout mice developed strong and comparable levels of proteinuria in response to the anti-GBM antibody (FIG. 4A). Interestingly, the expression of the cathepsin L cleavage targets synaptopodin and dynamin remained the same in wild type mice after induction of serum nephritis suggesting that development of proteinuria is independent of cathepsin L during serum nephritis (FIG. 4B). In contrast, detailed analysis of the kidney histology revealed significant differences in markers for renal disease progression. Podocyte apoptosis, crescent formation, glomerular sclerosis and glomerular hypercellularity were analyzed. Significantly more apoptotic podocyte nuclei were found in wild type mice with serum nephritis when compared to cathepsin L deficient animals with serum nephritis (FIG. 5A, insert). The same observation was supported by glomerular TUNEL staining. In addition, some glomeruli showed prominent crescent formation (FIG. 5B) which did not occur in cathepsin L knockout mice. All histological changes were semi-quantitated by analyzing glomeruli from different sections of the kidney (FIG. 5C). In conclusion, both wild type and cathepsin L knockout animals developed glomerular disease and comparable amounts of proteinuria but only wild type animals developed features of renal disease progression evidencing that the stability of CD2AP is directly related to the course of renal disease. Expression of cleavage resistant CD2AP halts renal disease progression: The absence of cathepsin L protected the expression of CD2AP and modified the degree of renal disease progression. The presence of N- and C-terminal CD2AP was analyzed in human kidney biopsies from patients with non-progressive glomerular disease (Minimal Change Disease, MCD) as well as from patients with progressive glomerular disease (Focal Segmental Glomerulosclerosis, FSGS), (FIG. 6A). While strong expression of N- and C-terminal CD2AP was found in normal and MCD glomeruli, a strong reduction of N-terminal CD2AP was observed in patients with FSGS. In contrast, the expression of C-terminal CD2AP was preserved arguing for a N-terminal degradation and presence of a C-terminal CD2AP fragment (FIG. 6A). To further analyze the effects of stable CD2AP on the course of progressive kidney disease, wild type CD2AP and CD2AP that is cleavage resistant against cathepsin L (FLAG-CD2AP-CatMut, FIG. 6B; FIG. 2G) were expressed. Equal expression of the two plasmids that both carry a FLAG tag, was monitored. The group of animals that received wild type CD2AP showed a significant reduction of N-terminal CD2AP but not the animals that expressed cleavage resistant CD2AP after serum nephritis (FIG. 6C). The expression of cleavage resistant CD2AP directly impacted on the severity of renal disease progression. Animals that expressed protected CD2AP developed significantly lower levels for podocyte apoptosis, crescent formation, glomerular sclerosis and glomerular hypercellularity (FIG. 6D, 6E). All together, the absence of cathepsin L (FIGS. 5A-5C) or the stable expression of CD2AP (FIG. 6A-6E) during serum nephritis alters the severity of renal disease progression.

Discussion: The data herein, provides an explanation for the importance of cathepsin L and CD2AP in the regulation of kidney podocyte survival and mechanistically links progression of renal disease with an enzymatic disease process within podocytes. How might these new findings being reconciled with earlier findings? Cathepsin L in the cytosol cleaves two important regulators of the podocyte actin cytoskeleton: 1) dynamin and 2) synaptopodin. In the case of dynamin, a N-terminal fragment is generated that possesses dominant-negative capabilities reorganizing the podocyte actin cytoskeleton. Synaptopodin proteolysis leads in turn to proteasomal degradation of RhoA. Both cleavage events can be inhibited by changes in target protein assembly; self-assembling into higher order dynamin complexes in the case of dynamin and phosphorylation dependent binding of 14-3-3 proteins to cover synaptopodin cleavage sites in the case of synaptopodin. The cleavage of these proteins results in the characteristic rearrangement of the podocyte actin cytoskeleton and the development of proteinuria. While these events can underlie the loss of barrier function, the cleavage of CD2AP helps to explain why loss of podocyte structure and function is often followed by podocyte depletion and progression of renal disease. In keeping with the hypothesis that bigenous heterozygosity for synaptopodin and CD2AP promotes the development of glomerulosclerosis in mice, this can also occur in acquired glomerular diseases via a cathepsin L-mediated enzymatic process. In essence, the genetic reduction (human haploinsufficiency) or absence of CD2AP (knockout) or the proteolysis of CD2AP in acquired diseases by cathepsin L provide situations in which the consequence will be loss of renal function. The data herein also identifies cathepsin L as instigator for both, proteinuria (e.g. through cleavage of dynamin and synaptopodin) and progression of renal disease through modification of CD2AP. This model suits well to explain the reversibility of glomerular disease such as Minimal Change Disease, where cathepsin L is induced but in smaller amounts than in FSGS or diabetic nephropathy4 and thus is associated with a stable CD2AP and the propensity to recover ad integrum. While dynamin, synaptopodin and CD2AP are cleavage targets of cytosolic cathepsin L, variances in susceptibility of the target proteins towards cathepsin L need to be defined in more detail to understand why not all proteinuric diseases with high cathepsin L progress and why not all glomerular diseases require cathepsin L for the development of proteinuria. Clearly, more future studies will be conducted to clarify these issues.

The enzymatic remodeling of CD2AP leads to a C-terminal fragment of CD2AP(p32) that is still capable to maintain some of its binding interactions and functions on the podocyte cytoskeleton and endocytosis but permits the release of its binding partner dendrin that can now travel to the podocyte nucleus to promote apoptosis and thus renal disease progression. The impact of this event becomes evident in the mouse serum nephritis model as well as by findings from the CD2AP^{-/-} mouse. Both animals display nuclear relocation of dendrin and both animal models have progressive renal disease. Full length CD2AP executing its SH3 binding adapter capabilities is required for sustained podocyte survival even in the presence of proteinuria.

PEPS-computer simulation predicted eleven cathepsin L cleavage sites in CD2AP amino acid sequence but only two were experimentally confirmed. Although PEPS is based on cleavage sites within native proteins, it does no further adjustment for secondary or tertiary protein structures that may sterically hinder the access of the protease to the cleavage site in a candidate substrate such as CD2AP. Hence, experimental validation of the putative cleavage sites is required. Thus, the experiments conducted herein included both structural and biochemical studies. Negative stain electron microscopy of purified CD2AP revealed uniform particles with a size and morphology suggesting a tetrameric organization, verified with chemical crosslinking. Single particle image analysis was used to generate a 3-D map of the cuboid CD2AP tetramer. Many of the computer-modeled cleavage sites are inaccessible due to tetramerization of CD2AP but the two sites QPLGS and LSAAE. Both sites are located at the SH3 arms of CD2AP that allows access by cathepsin L and thus provide starting points for cathepsin L remodeling of CD2AP. It is interesting that the deletion of the LSAAE site is sufficient to inhibit the enzymatic processing suggesting that cleavage at this site may occur first.

While synaptopodin is known to bind to the SH3 domains of CD2AP, it was surprising to see that it retains binding capacity to p32. This is best explained through a cryptic binding site that allows synaptopodin binding to p32 fragment of CD2AP. In contrast, this binding site is not sufficient to maintain dendrin binding. While these structural studies are necessary to better define the cleavage process and its consequences, they also help to better define the role of CD2AP in the hereditary form of FSGS in families with CD2AP mutation. A C-terminal stop mutation will lead to a deformation of the length of the CD2AP coiled-coil domain and will inhibit actin binding to CD2AP. The structure of CD2AP will also provide starting points to better understand its function in cytoskeletal regulation in general, e.g. in T-cell polarity as well as identify possible other tissues where CD2AP might be regulated by proteolysis.

While cathepsin L has been identified to be causative for LPS and PAN-mediated proteinuria, serum nephritis is a glomerular disease that does not require cathepsin L for proteinuria yet for the progression of renal disease. It is intriguing that stabilization of CD2AP by removing cathepsin L or protecting CD2AP alters the course of a glomerular disease shifting progression into a more benign phenotype. This finding will allow for the development of additional strategies for renal protection that are in addition to anti-proteinuric modalities focusing on podocyte survival.

Although the invention has been illustrated and described with respect to one or more implementations, equivalent alterations and modifications will occur to others skilled in the art upon the reading and understanding of this specification and the annexed drawings. In addition, while a particular feature of the invention may have been disclosed with respect to only one of several implementations, such feature may be combined with one or more other features of the other implementations as may be desired and advantageous for any given or particular application.

The Abstract of the disclosure will allow the reader to quickly ascertain the nature of the technical disclosure. It is submitted with the understanding that it will not be used to interpret or limit the scope or meaning of the following claims.

What is claimed is:

1. A method of treating renal diseases or disorders, comprising administering to a patient in need thereof, an effective amount of an agent which inhibits cytoskeletal adaptor protein (CD2AP) degradation and/or modulates expression or activity of CD2AP and/or modulates cathepsin-L expression or activity in vivo and,
treating renal diseases or disorders.

2. The method of claim 1, wherein the renal diseases or disorders comprising: podocyte diseases or disorders, proteinuria, glomerular diseases, membranous glomerulonephritis, focal segmental glomerulonephritis, minimal change disease, nephrotic syndromes, pre-eclampsia, eclampsia, kidney lesions, collagen vascular diseases, stress, strenuous exercise, benign orthostatic (postural) proteinuria, focal segmental glomerulosclerosis (FSGS), IgA nephropathy, IgM nephropathy, membranoproliferative glomerulonephritis, membranous nephropathy, sarcoidosis, Alport's syndrome, diabetes mellitus, kidney damage due to drugs, Fabry's disease, infections, aminoaciduria, Fanconi syndrome, hypertensive nephrosclerosis, interstitial nephritis, Sickle cell disease, hemoglobinuria, multiple myeloma, myoglobinuria, Wegener's Granulomatosis or Glycogen Storage Disease Type 1.

3. The method of claim 1, wherein an inhibitor of cathepsin L comprises: nucleic acids, cathepsin L mutants, CD2AP mutants, oligonucleotides, polynucleotides, peptides, polypeptides, antibodies, small molecules, organic or inorganic molecules.

4. The method of claim 3, wherein a CD2AP mutant is resistant to degradation by cathepsin L, or other enzyme.

5. The method of claim 3, wherein a CD2AP mutant comprises mutations in amino acid sequences susceptible to cathepsin L activity comprising: ELRKE (SEQ ID NO: 1), ELAKA (SEQ ID NO: 2), LPGRF (SEQ ID NO: 3), AFVAR (SEQ ID NO: 4), LSAAE (SEQ ID NO: 5), ELGKE (SEQ ID NO: 6), QPLGS (SEQ ID NO: 7), KIRGI (SEQ ID NO: 8), APGSV (SEQ ID NO: 9), LIVGV (SEQ ID NO: 10), EIIRV (SEQ ID NO: 11), mutants, derivatives, variants or combinations thereof.

6. The method of claim 1, wherein an antibody specific for cathepsin L cytoskeletal adaptor protein (CD2AP) cleavage sites block or inhibit cathepsin L degradation of CD2AP.

7. The method of claim 1, wherein an agent for modulating expression, function and/or activity of CD2AP in vivo, comprising at least one of: antibody, aptamer, antisense oligonucleotide, polynucleotides, enzymes, peptides, polypeptides, organic or inorganic molecules.

8. A method of identifying agents which modulate cathepsin-L expression, function and/or activity in vivo comprising:
culturing a kidney cell or kidney cell line;
contacting said cells with one or more agents;
measuring the cathepsin-L activity in podocytes; and,
identifying agents which modulate the cathepsin-L expression, function and/or activity in vivo.

9. The agent of claim 8, wherein the agent decreases cathepsin L activity or expression in vivo and/or inhibits cytoskeletal adaptor protein (CD2AP) degradation as compared to normal controls.

10. A method of identifying agents which modulate cytoskeletal adaptor protein (CD2AP) degradation, expression, function and/or activity comprising:
culturing a kidney cell or kidney cell line;
contacting said cells with one or more agents;
measuring the cytoskeletal adaptor protein (CD2AP) degradation, expression, function, or activity; and,
identifying agents which modulate cytoskeletal adaptor protein (CD2AP) degradation, expression, function and/or activity.

11. The method of claim 10, wherein the cytoskeletal adaptor protein (CD2AP) degradation is inhibited by an agent by at least 10% as compared to a normal control.

12. The method of claim 10, wherein the cytoskeletal adaptor protein (CD2AP) degradation is inhibited by an agent by at least about 50% as compared to a normal control.

13. The method of claim 10, wherein the cytoskeletal adaptor protein (CD2AP) degradation is inhibited by an agent by 100% as compared to a control.

14. The method of claim 10, wherein the agent further inhibits rate of degradation of cytoskeletal adaptor protein (CD2AP) as compared to a normal control.

15. The method of claim 10, wherein the agent increases CD2AP expression, function and/or activity by at least about 1 fold as compared to a normal control.

16. The method of claim 10, wherein the agent increases CD2AP expression, function and/or activity by at least about 5 fold as compared to a normal control.

17. The method of claim 10, wherein the agent increases CD2AP expression, function and/or activity up to 1000 fold as compared to a normal control.

18. A cathepsin resistant cytoskeletal adaptor protein (CD2AP) molecule comprising a mutation at one or more amino acids in a cathepsin L cleavage site.

19. The cathepsin resistant CD2AP molecule of claim 18, wherein the cathepsin cleavage site comprises the amino acid sequences set forth as ELRKE (SEQ ID NO: 1), ELAKA (SEQ ID NO: 2), LPGRF (SEQ ID NO: 3), AFVAR (SEQ ID NO: 4), LSAAE (SEQ ID NO: 5), ELGKE (SEQ ID NO: 6), QPLGS (SEQ ID NO: 7), KIRGI (SEQ ID NO: 8), APGSV (SEQ ID NO: 9), LIVGV (SEQ ID NO: 10), EIIRV (SEQ ID NO: 11), mutants, derivatives, variants or combinations thereof.

20. The cathepsin resistant CD2AP molecule of claim 18, wherein a cathepsin cleavage site mutant comprises a sequence similarity to SEQ ID NOS: 1 to 11 of between about 5% to 99.99% sequence similarity.

21. The cathepsin resistant CD2AP molecule of claim 18, wherein a cathepsin cleavage site mutant comprises an amino acid sequence of between about 1 amino acid to about 15 amino acids.

22. The cathepsin resistant CD2AP molecule of claim 18, wherein the CD2AP molecule lacks one or more amino acids in the sequences set forth as SEQ ID NOS: 1 to 11.

23. A composition comprising a pharmaceutical composition and/or one or more cathepsin L inhibitors and/or agents which inhibit cytoskeletal adaptor protein (CD2AP) degradation, in a therapeutically effective amount.

24. A composition comprising an agent which increases expression, function, and/or activity of cytoskeletal adaptor protein (CD2AP), in a therapeutically effective amount.

25. A vector expressing a cytoskeletal adaptor protein (CD2AP) cathepsin L resistant molecule.

26. A biomarker for the diagnosis of a disease or disorder characterized by proteinuria and/or identification of individuals at risk of developing a disease or disorder characterized by proteinuria comprising: cathepsin-L, dynamin, synaptopodin or cytoskeletal regulator protein synaptopodin, cytoskeletal adaptor protein (CD2AP), variants, mutants or fragments thereof.

27. The biomarker of claim 26, wherein a fragment of CD2AP comprises p32 C-terminal fragment.

28. The biomarker of claim 26, wherein expression of dendrin is increased in podocyte nuclei.

29. The biomarker of claim 26, wherein the identification of an individual at risk of developing disease or disorder characterized by proteinuria detects at least one biomarker or fragments thereof.

30. The biomarker of claim 26, wherein the progression of disease or disorder characterized by proteinuria is correlated to an increase in cathepsin-L and/or system N glutamine transporter (SNAT3) expression.

31. The biomarker of claim 26, wherein the progression of disease or disorder characterized by proteinuria is correlated to an increase in p32 CD2AP C-terminal fragment expression and/or dendrin in podocyte nuclei.

32. An antibody or aptamer specific for CD2AP, mutants, variants, fragments, derivatives or analogs thereof.

33. The antibody or aptamer of claim 32, wherein at least one antibody specifically binds to ELRKE (SEQ ID NO: 1), ELAKA (SEQ ID NO: 2), LPGRF (SEQ ID NO: 3), AFVAR (SEQ ID NO: 4), LSAAE (SEQ ID NO: 5), ELGKE (SEQ ID NO: 6), QPLGS (SEQ ID NO: 7), KIRGI (SEQ ID NO: 8), APGSV (SEQ ID NO: 9), LIVGV (SEQ ID NO: 10), EIIRV (SEQ ID NO: 11), mutants, derivatives, variants or combinations thereof.


SEQUENCE LISTING (12 sequences)

SEQ ID NO: 1 - protein, 5 aa, Artificial Sequence
  Description of Artificial Sequence Synthetic peptide
ELRKE

SEQ ID NO: 2 - protein, 5 aa, Artificial Sequence
  Description of Artificial Sequence Synthetic peptide
ELAKA

SEQ ID NO: 3 - protein, 5 aa, Artificial Sequence
  Description of Artificial Sequence Synthetic peptide
LPGRF

SEQ ID NO: 4 - protein, 5 aa, Artificial Sequence
  Description of Artificial Sequence Synthetic peptide
AFVAR

SEQ ID NO: 5 - protein, 5 aa, Artificial Sequence
  Description of Artificial Sequence Synthetic peptide
LSAAE

SEQ ID NO: 6 - protein, 5 aa, Artificial Sequence
  Description of Artificial Sequence Synthetic peptide
ELGKE

SEQ ID NO: 7 - protein, 5 aa, Artificial Sequence
  Description of Artificial Sequence Synthetic peptide
QPLGS

SEQ ID NO: 8 - protein, 5 aa, Artificial Sequence
  Description of Artificial Sequence Synthetic peptide
KIRGI

SEQ ID NO: 9 - protein, 5 aa, Artificial Sequence
  Description of Artificial Sequence Synthetic peptide
APGSV

SEQ ID NO: 10 - protein, 5 aa, Artificial Sequence
  Description of Artificial Sequence Synthetic peptide
LIVGV

SEQ ID NO: 11 - protein, 5 aa, Artificial Sequence
  Description of Artificial Sequence Synthetic peptide
EIIRV

SEQ ID NO: 12 - protein, 4 aa, Artificial Sequence
  Description of Artificial Sequence Synthetic peptide
RKLW